(19) United States
(12) Patent Application Publication
(10) Pub. No.: US 2016/0081625 A1
(43) Pub. Date: Mar. 24, 2016

(54) METHOD AND APPARATUS FOR PROCESSING SENSOR DATA

(71) Applicant: Samsung Electronics Co., Ltd., Gyeonggi-do (KR)

(72) Inventors: Jin-Ik KIM, Seoul (KR); Sang-Chul KU, Gyeonggi-do (KR); Sun-Kyung PARK, Gyeonggi-do (KR); Su-Ha YOON, Seoul (KR); Yo-Han LEE, Gyeonggi-do (KR)

(21) Appl. No.: 14/856,915

(22) Filed: Sep. 17, 2015

(30) Foreign Application Priority Data
Sep. 23, 2014 (KR) 10-2014-0126938

(51) Int. Cl. A61B 5/00; A61B 5/0402; A61B 5/0205; H04W 4/00

(52) U.S. Cl. CPC A61B 5/6898; H04W 4/008; H04W 4/005; A61B 5/0402; A61B 5/0205; A61B 5/7264; A61B 2562/0219; A61B 2562/0223

(57) ABSTRACT

A method in an electronic device is provided, the method including: receiving sensor data from at least one external electronic device; determining an exercise type of a user who wears the at least one external electronic device based on the received sensor data; and outputting exercise information corresponding to the determined exercise type.

### CLAM OF PRIORITY

This present application is related to and claims the benefit under 35 U.S.C. §119(a) of a Korean patent application No. 10-2014-0126938 filed in the Korean Intellectual Property Office on Sep. 23, 2014, the entire disclosure of which is hereby incorporated by reference.

### BACKGROUND

1. Field of the Disclosure

The present disclosure relates to electronic device and more particularly to a method and apparatus for processing sensor data.

2. Description of Related Art

With the advance of information communication techniques and semiconductor techniques, various electronic devices are being developed into multimedia devices for providing various multimedia services. For example, the electronic device may provide various multimedia services such as a messenger service, a broadcast service, a wireless Internet service, a camera service, and a music play service. As a health is emphasized in modern society, the electronic device provides a health care service for measuring an exercise amount, a blood sugar, or the like. For example, a user can check a current exercise amount or may check health information such as a heart rate or the like during exercising.

### SUMMARY

According to various embodiments of the present disclosure, a sensor data processing method for synchronizing sensor data received from at least one external electronic device, and an electronic device thereof are provided.

According to various embodiments of the present disclosure, a sensor data processing method for analyzing exercise information based on an attribute of sensor data, and an electronic device thereof are provided.

According to various embodiments of the present disclosure, a sensor data processing method for outputting exercise information corresponding to an exercise type, and an electronic device thereof are provided.

According to various embodiments of the present disclosure, a method of operating an electronic device may include receiving sensor data from at least one external electronic device, determining an exercise type of a user who wears the at least one external electronic device based on the received sensor data, and outputting exercise information corresponding to the determined exercise type.

According to various embodiments of the present disclosure, an electronic device may include a communication module, and a processor operatively coupled to the communication module, wherein the processor controls to receive sensor data from at least one external electronic device through the communication module, determine an exercise type of a user who wears the at least one external electronic device based on the received sensor data, and output exercise information corresponding to the determined exercise type.

According to various implementations of the present disclosure, an electronic device may include a memory storing mobility data for a plurality of activity types,

a communication interface configured to receive sensor data from at least one external electronic device, and at least one processor configured to determine a particular one of the plurality of activity types by comparing the sensor data from the at least one external device or processed sensor data from the at least one external device to the mobility data for the plurality of activity types.

### BRIEF DESCRIPTION OF THE DRAWINGS

The above and other aspects, features and advantages of various aspects of the present disclosure will be more apparent from the following detailed description taken in conjunction with the accompanying drawings, in which:

FIG. 1 is a block diagram of an electronic device according to an embodiment of the present disclosure;

FIG. 2 is a block diagram of a sensor data processing module according to an embodiment of the present disclosure;

FIG. 3 is a flowchart for a method in which an electronic device outputs exercise information based on sensor data of at least one external electronic device according to an embodiment of the present disclosure;

FIG. 4 is a flowchart of a method in which an electronic device synchronizes sensor data of at least one external electronic device according to an embodiment of the present disclosure;

FIG. 5 and FIG. 6 illustrate a method of performing synchronization based on synchronization related information according to an embodiment of the present disclosure;

FIG. 7A, FIG. 7B and FIG. 7C illustrate synchronization of data of at least one sensor according to an embodiment of the present disclosure;

FIG. 8 is a flowchart of a method in which an electronic device receives synchronized sensor data from an external electronic device according to an embodiment of the present disclosure;

FIG. 9 is a flowchart of a method in which an electronic device analyzes sensor data of at least one external electronic device according to an embodiment of the present disclosure;

FIG. 10 illustrates a method of distinguishing static sensor data or dynamic sensor data based on a location of an external electronic device according to an embodiment of the present disclosure;

FIG. 11 illustrates a screen configuration for determining a location of a sensor of an external electronic device according to an embodiment of the present disclosure;

FIG. 12 illustrates a pattern of sensor data corresponding to an exercise type according to an embodiment of the present disclosure;

FIG. 13A, FIG. 13B, FIG. 14, FIG. 15, FIG. 16, FIG. 17A, FIG. 17B, FIG. 18, FIG. 19A, FIG. 19B, FIG. 20A and FIG. 20B illustrate a method of providing exercise information through at least one sensor according to an embodiment of the present disclosure;

FIG. 21 illustrates a method of controlling an electronic device through at least one sensor according to an embodiment of the present disclosure;

FIG. 22A and FIG. 22B illustrate a method of providing driving information (e.g., for distinguishing a driver) through at least one sensor according to an embodiment of the present disclosure;

FIG. 23 illustrates a method of determining a localized motion through at least one sensor according to an embodiment of the present disclosure;

FIG. 24A, FIG. 24B, FIG. 25 and FIG. 26 illustrate a method of outputting exercise information (feedback) according to an embodiment of the present disclosure;

FIG. 27 is a flowchart of a method in which an electronic device synchronizes sensor data of at least one external electronic device and analyzes the synchronized sensor data according to an embodiment of the present disclosure;

FIG. 28A and FIG. 28B illustrate a calibration operation of a sensor according to an embodiment of the present disclosure;

FIG. 29 is a flowchart of a method in which an electronic device synchronizes sensor data of at least one external electronic device and analyzes the synchronized sensor data according to an embodiment of the present disclosure; and

FIG. 30 illustrates a block diagram of an electronic device according to various embodiments of the present disclosure.

### DETAILED DESCRIPTION

Hereinafter, various embodiments of the present disclosure will be described with reference to the accompanying drawings. The present disclosure may be modified in various forms and include various embodiments, but specific examples are illustrated in the drawings and described in the description. However, the description is not intended to limit the present disclosure to the specific embodiments, and it shall be appreciated that all the changes, equivalents and substitutions belonging to the idea and technical scope of the present disclosure are included in the present disclosure. In the description of the drawings, identical or similar reference numerals are used to designate identical or similar elements.

The term “include” or “may include” refers to the existence of a corresponding disclosed function, operation or component which can be used in various embodiments of the present disclosure and does not limit one or more additional functions, operations, or components. In the present disclosure, the terms such as “include” or “have” may be construed to denote a certain characteristic, number, step, operation, constituent element, component or a combination thereof, but may not be construed to exclude the existence of or a possibility of addition of one or more other characteristics, numbers, steps, operations, constituent elements, components or combinations thereof.

In various embodiments of the present disclosure, the expression “or” or “at least one of A or/and B” includes any or all of combinations of words listed together. For example, the expression “A or B” or “at least A or/and B” may include A, may include B, or may include both A and B.

The expression “1”, “2”, “first”, or “second” used in various embodiments of the present disclosure may modify various components of various embodiments but does not limit the corresponding components. For example, the above expressions do not limit the sequence and/or importance of the elements. The above expressions are used merely for the purpose of distinguishing an element from the other elements. For example, a first electronic device and a second electronic device indicate different electronic devices although both of them are electronic devices. For example, without departing from the scope of the present disclosure, a first component element may be named a second component element. Similarly, the second component element also may be named the first component element.

In the case where an element is referred to as being “connected” or “accessed” to other elements, it should be understood that not only the element is directly connected or accessed to the other elements, but also another element may exist between them. Meanwhile, in the case where an element is referred to as being “directly connected to” or “directly accessing” other elements, it should be understood that there is no element therebetween.

The terms in various embodiments of the present disclosure are used to describe a specific embodiment, and are not intended to limit the present disclosure. As used herein, the singular forms are intended to include the plural forms as well, unless the context clearly indicates otherwise.

Unless defined differently, all terms used herein, which include technical terminologies or scientific terminologies, have the same meaning as a person skilled in the art to which the present disclosure belongs. Such terms as those defined in a generally used dictionary are to be interpreted to have the meanings equal to the contextual meanings in the relevant field of art, and are not to be interpreted to have ideal or excessively formal meanings unless clearly defined in the present disclosure.

An electronic device according to various embodiments of the present disclosure may be a device with a communication function. For example, the electronic device may include at least one of a smart phone, a tablet Personal Computer (PC), a mobile phone, a video phone, an e-book reader, a desktop PC, a laptop PC, a netbook computer, a PDA, a Portable Multimedia Player (PMP), an MP3 player, a mobile medical device, a camera, a wearable device (for example, a Head-Mounted-Device (HMD) such as electronic glasses, electronic clothes, an electronic bracelet, an electronic necklace, an electronic appcessory, an electronic tattoo, and or a smart watch.

According to some implementations, the electronic device may be a smart home appliance with a communication function. The smart home appliance as an example of the electronic device may include at least one of a television, a Digital Video Disk (DVD) player, an audio, a refrigerator, an air conditioner, a vacuum cleaner, an oven, a microwave oven, a washing machine, an air cleaner, a set-top box, a TV box (e.g., Samsung HomeSync™, Apple TV™, or Google TV™), a game console, an electronic dictionary, an electronic key, a camcorder, and or an electronic picture frame.

According to some implementations, the electronic device may include at least one of various types of medical devices (for example, Magnetic Resonance Angiography (MRA), Magnetic Resonance Imaging (MRI), Computed Tomography (CT), a scanning machine, ultrasonic wave device and the like), a navigation device, a Global Positioning System (GPS) receiver, an Event Data Recorder (EDR), a Flight Data Recorder (FDR), a car infotainment device, ship electronic equipment (for example, navigation equipment for a ship, a gyro compass and the like), avionics, a security device, or and an industrial or home robot.

According to another implementation, the electronic devices may include at least one of furniture or a part of a building/structure having a communication function, electronic boards, electronic signature receiving devices, proj ectors, or various measuring equipment (e.g., equipment for a water supply, an electricity, gases or radio waves).

An electronic device according to various implementations of the present disclosure may be a combination of one or more of above described various devices. Also, an electronic device according to various implementations of the present disclosure may be a flexible device. Also, an electronic device according to various implementations of the present disclosure is not limited to the above described devices.

Hereinafter, an electronic device according to various implementations will be described with reference to the accompanying drawings. The term “user” used in various implementations may refer to a person who uses an electronic device or a device (for example, an artificial intelligence electronic device) that uses an electronic device.

According to certain implementations, detailed operation and posture of an exercise currently performed in addition to whether the user is walking or running or riding a bicycle or the like is simply classified by the electronic device, and an exercise amount on the basis thereof is reported.

According to various implementations of the present disclosure, a method of operating an electronic device may include receiving sensor data from at least one external electronic device, determining an exercise type of a user who wears the at least one external electronic device based on the received sensor data, and outputting exercise information corresponding to the determined exercise type.

According to various implementations, the sensor data may include at least one of acceleration information, angular velocity information, rotation information, geomagnetic field information, and electrocardiogram information.

According to various implementations, the method may further include receiving synchronization related information from the at least one external electronic device.

According to various implementations, the method may further include synchronizing the sensor data received from the at least one external electronic device based on the synchronization related information.

According to various implementations, the synchronization related information may include at least one of time stamp information, time index information, and a sampling rate of a sensor of the at least one external electronic device.

According to various implementations, the determining of the exercise type may include classifying a mobility feature of the sensor data, analyzing the sensor data according to the classified mobility feature, and determining the exercise type according to a result of the analysis.

According to various implementations, the mobility feature of the sensor data may be determined according to a location on which the at least one external electronic device is worn, a type of the at least one external electronic device, or an input of the user.

According to various implementations, the determining of the exercise type may include confirming a pattern of the sensor data, comparing the confirmed pattern with a pre-stored pattern, and determining the exercise type according to a result of the comparison.

According to various implementations, the outputting of the exercise information may include outputting posture information, exercise count information, heart rate information, speed information, and distance information based on the exercise type.

According to various implementations, the method may further include, after the determining of the exercise type, adjusting at least one of a sampling rate, a transmission rate of the sensor data, and a transmission period of the sensor data according to the determined exercise type.

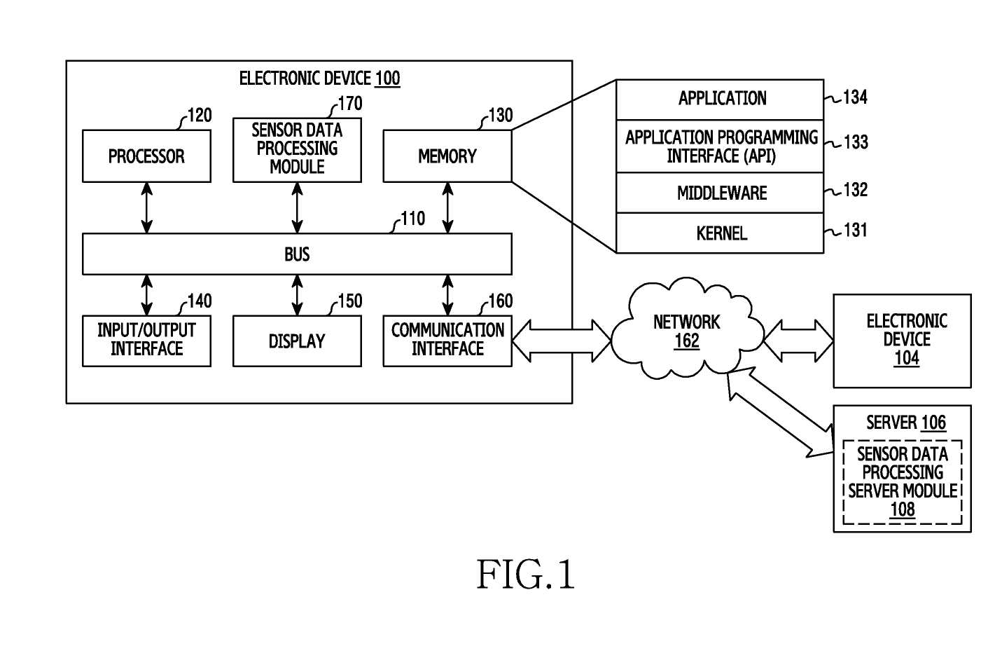

FIG. 1 is a block diagram of an electronic device according to an embodiment of the present disclosure.

Referring to FIG. 1, the electronic device 100 may include a bus 110, a processor 120, a memory 130, an input/output interface 140, a display 150, a communication interface 160, and a sensor data processing module 170. According to an embodiment, the sensor data processing module 170 may be included in the processor 120 to operate or may be included in a separate module to interwork with the processor 120.

The bus 110 may be a circuit that interconnects the above-described components and delivers communications (for example, a control message) between the above-described components.

The processor 120 may, for example, receive a command from other components (for example, the memory 130, the input/output interface 140, the display 150, the communication interface 160, the sensor data processing module 170, etc.), through the bus 110, may decrypt the received command, and may execute operation or data processing based on the decrypted command.

The memory 130 may store a command or data received from the processor 120 or other components (for example, the input/output interface 140, the display 150, the communication interface 160, the sensor data processing module 170, and the like), or generated by the processor 120 or other components.

The memory 130 may include programming modules, for example, a kernel 131, a middleware 132, an Application Programming Interface (API) 133, an application 134, and the like. Each of the aforementioned programming modules may be formed of software, firmware, hardware, or a combination of at least two thereof.

According to an embodiment, the kernel 131 may control or manage system resources, for example, the bus 110, the processor 120, the memory 130, and the like, used for executing an operation or function implemented in other programming modules, for example, the middleware 132, the API 133, or the applications 134. Also, the kernel 131 may provide an interface that enables the middleware 132, the API 133, or the applications 134 to access an individual component of the electronic device 100 for control or management.

According to an embodiment, the middleware 132 may execute operate as a relay so that the API 133 or the applications 134 communicates to exchange data with the kernel 131. Also, in association with operation requests received from the application 134, the middle ware 132 may execute a control, for example, scheduling or load balancing, for an operation request, through use of, for example, a method of assigning, to at least one of application 134, a priority of use of a system resource of the electronic device 100, for example, the bus 110, the processor 120, the memory 130, or the like).

According to an embodiment, the API 133 is an interface used by the applications 134 to control a function provided from the kernel 131 or the middleware 132, and may include, for example, at least one interface or function, for example, an instruction, for a file control, a window control, image processing, a character control, or the like.

According to an embodiment, the applications 134 may include a Short Message Service (SMS)/Multimedia Message Service (MMS) application, an e-mail application, a calendar application, an alarm application, a health care application (for example, an application for measuring a work rate or a blood sugar), an environment information application (for example, an application for providing atmospheric pressure, humidity, or temperature information). Additionally or alternatively, the application 134 may be an application associated with exchanging of information between the electronic device 100 and an external electronic device (for example, an electronic device 104). The application related to the information exchange may include, for example, a notification transmission application for transferring predetermined information to an external electronic device or a device management application for managing an external electronic device.

For example, the notification relay application may include a function of transferring, to the external electronic device, for example, the electronic device 104, notification information generated from other applications of the electronic device 100, for example, an SMS/MMS application, an e-mail application, a health management application, an environmental information application, and the like. Additionally or alternatively, the notification relay application may receive notification information from, for example, an external electronic device (for example, the electronic device 104), and may provide the notification information to a user. For example, the device management application may manage (for example, install, delete, or update) a function for at least some parts of the external electronic device (for example, the electronic device 104) communicating with the electronic device 100 (for example, a function of turning on/off the external electronic device itself, (or some components,) or a function of adjusting luminance (or a resolution) of the display), applications operating in the external electronic device, or services provided by the external electronic device (for example, a call service and a message service).

According to various embodiments, the applications 134 may include an application designated based on properties (for example, a type of electronic device) of an external electronic device (for example, the electronic device 104). For example, when the external electronic device is an MP3 player, the application 134 may include an application related to the reproduction of music. Similarly, when the external electronic device is a mobile medical device, the application 134 may include an application related to health care. According to an one embodiment, the applications 134 may include at least one of applications received from an application designated for the electronic device 100 or an application received from an external electronic device (for example, a server 106 or the electronic device 104).

According to an embodiment, the input/output interface 140 may transfer a command or data input by a user through an input/output device (for example, a sensor, a keyboard, or a touch screen) to the processor 120, the memory 130, the communication interface 160, and the sensor data processing module 170, for example, through the bus 110. For example, the input/output interface 140 may provide, to the processor 120, data associated with a touch of a user input through a touch screen. Further, the input/output interface 140 may output, for example, command or data received through the bus 110 from the processor 120, the memory 130, the communication interface 160, and the sensor data processing module 170, to an input/output device (for example, a speaker or display). For example, the input/output interface 140 may output voice data processed by the processor 120 to the user through a speaker.

According to one embodiment, the display 150 may display various pieces of information (for example, multimedia data, text data, and the like) to a user.

According to one embodiment, the communication interface 160 may connect communication between the electronic device 100 and an electronic device (for example, the electronic device 104 or the server 106). For example, the communication interface 160 may be connected to the network 162 through wireless communication or wired communication, and may communicate with an external device. The wireless communication may include at least one of, for example, Wi-Fi, Bluetooth (BT), Near Field Communication (NFC), Global Positioning System (GPS) or and cellular communication (for example LTE, LTE-A, CDMA, WCDMA, UMTS, WiBro, GSM, etc.). The wired communication may include at least one of, for example, a Universal Serial Bus (USB), a High Definition Multimedia Interface (HDMI), a Recommended Standard 232 (RS-232), or and a Plain Old Telephone Service (POTS).

According to one embodiment, the network 162 may be a communication network. The telecommunication network may include at least one of a computer network, Internet, Internet of things, or and a telephone network. According to an one embodiment, a protocol (for example, a transport lay protocol, data link layer protocol, or a physical layer protocol) for communication between the electronic device 100 and the external device may be supported by at least one of the applications 134, the application programming interface 133, the middleware 132, the kernel 131, or and the communication interface 160.

According to one embodiment, the sensor data processing module 170 may receive sensor data from at least one external electronic device, and may determine an exercise type of a user who wears the external electronic device based on the received sensor data. According to one embodiment, the sensor data processing module 170 may collect and synchronize the received sensor data, and may analyze the synchronized sensor data. According to one embodiment, the sensor data processing module 170 may output exercise information corresponding to the determined exercise type, and may analyze the sensor data and thereafter perform a specific function. An operation of the sensor data processing module 170 will be described below in greater detail.

According to one embodiment, the server 106 may support driving of the electronic device 100 by performing at least one of operations (or functions) implemented in the electronic device 100. For example, the server 106 may include a sensor data processing server module 108 capable of supporting the sensor data processing module 170 implemented in the electronic device 100. According to one embodiment, the sensor data processing server module 108 may include at least one constitutional elements of the sensor data processing module 170, and may perform (e.g., replace) at least one of operations performed by the sensor data processing module 170.

According to one embodiment, the sensor data processing module 170 may process at least one part of information acquired from different constitutional elements (e.g., the processor 120, the memory 130, the input/output interface 140, the communication interface 160, or the like), and may provide this to a user in various manners. For example, the sensor data processing module 170 may control at least some functions of the electronic device 100 independently or by using the processor 120 so that the electronic device 100 interworks with another electronic device (e.g., the electronic device 104 or the server 106). According to one embodiment, at least one constitutional element of the sensor data processing module 170 may be included in the server 106 (e.g., the sensor data processing server module 108), and may be supported with at least one operation implemented in the sensor data processing module 170.

According to certain implementations, the memory 130 can store dynamic and static mobility sensor data that is commonly associated with a variety of activities. The application 134 can comprise a plurality of executable instructions that are executable by either the processor 120, the sensor data processing module 170, or a combination thereof. The term “one or more processors” shall now be understood to refer to a processor, such as processor 120, a sensor data processing module, such as sensor data processing module 170, or a combination thereof. When the plurality of executable instructions are executed by the one or more processors, the instructions cause the any of the one or more processors to control the communication interface 160 to receive sensor data from the electronic device 104. The one or processors can determine an exercise type of a user who wears electronic device 104. The one or more processors can then control the display 150 to output exercise information corresponding to the determined exercise type.

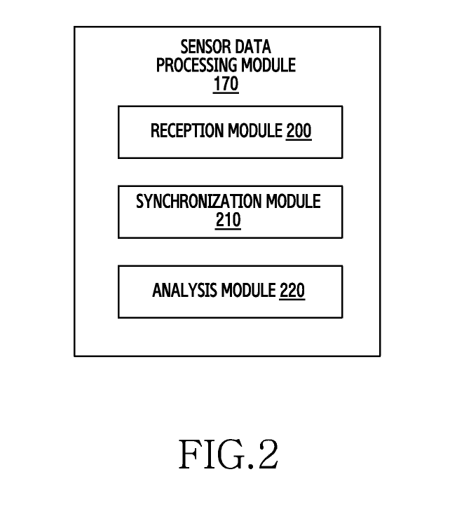

FIG. 2 is a block diagram of a sensor data processing module according to an embodiment of the present disclosure.

Referring to FIG. 2, the sensor data processing module 170 may include a reception module 200, a synchronization module 210, and an analysis module 220. According to one embodiment, the sensor data processing module 170 may further include an extra module in addition to the aforementioned modules.

According to one embodiment, the reception module 200 may receive sensor data from at least one external electronic device. For example, the sensor data may include a sensor value acquired from a sensor of the external electronic device. The sensor data may include sensor data which indicates a user's posture, motion state (e.g., standstill, walking, running, stair-walking, etc.), a specific health state (e.g., a heart rate), and a specific situation (e.g., daytime, nighttime, indoor, outdoor, flooded, etc.). For example, the sensor data may include a variety of information such as acceleration information, angular velocity information, rotation information, geomagnetic field information, heart rate information, or the like.

According to one embodiment, the reception module 200 may receive synchronization related information from an external electronic device. According to one embodiment, the synchronization related information may be time stamp information or time index information of a corresponding device. For example, the time stamp information or the time index information may include clock related information or an index value or the like of a current clock of a corresponding device. According to one embodiment, the synchronization related information may include a sampling rate, data format information, manufacturer information, or Operating System (OS) information of a corresponding sensor. For example, the synchronization related information may have various sampling rates of sensors of respective devices, and may include data format information, data unit information, or coordinate system information based on X, Y, and Z axes of each sensor.

According to one embodiment, the synchronization module 210 may synchronize sensor data. According to one embodiment, the synchronization module 210 may synchronize the sensor data based on the synchronization related information. According to one embodiment, the synchronization module 210 may perform synchronization by utilizing a clock index value difference between respective external electronic devices. For example, the synchronization module 210 may calculate an average value by receiving a clock value several times from the external electronic device, and thereafter may perform synchronization based on the average value. According to one embodiment, the synchronization module 210 may perform synchronization based on a sampling rate of a sensor of each external electronic device. For example, the synchronization module 210 may collect a sampling rate of at least one external electronic device, and may compare and analyze the collected sampling rate to perform synchronization of the sensor data. According to one embodiment, the synchronization module 210 may perform calibration of the sensor data. For example, the synchronization module 210 may perform a calibration operation before analyzing the sensor data. In addition, the synchronization module 210 may perform the calibration operation when a user is in a standstill state.

According to one embodiment, the analysis module 220 may determine an exercise type by analyzing the synchronized sensor data. According to one embodiment, the analysis module 220 may analyze data by utilizing the synchronized sensor data and mobility information of each sensor. The analysis module 220 may determine a user's exercise type based on the analyzed data. According to one embodiment, the analysis module 220 may distinguish a mobility of each sensor of at least one external electronic device. According to one embodiment, each sensor may have a static mobility or dynamic mobility feature according to the mobility. For example, each sensor may have a different mobility according to a location of the sensor, and a location of the sensor may be determined according to a device type. According to one embodiment, the location of the sensor may be determined by a user input. According to one embodiment, the analysis module 220 may determine the current exercise type by using a pattern of the sensor data. For example, an electronic device may have a pattern of sensor data for each exercise, and the pattern of sensor data may be classified into dynamic sensor data and static sensor data. According to one embodiment, in case of a tennis, first sensor data acquired from an external electronic device worn on a head may be classified as dynamic sensor data, and second sensor data acquired from an external electronic device worn on a wrist may be classified as static sensor data.

According to one embodiment, the analysis module 220 may perform a specific function after analyzing the sensor data. According to one embodiment, the analysis module 220 may adjust a sampling rate for the determined exercise type after analyzing the sensor data. For example, the analysis module 220 may increase a sampling rate in a speedy exercise such as a tennis, and may decrease the sampling rate as to a relatively slow exercise such as jogging. According to one embodiment, the analysis module 220 may adjust a data transfer rate after analyzing the sensor data. For example, the analysis module 220 may provide control such that only a part of the sensor data is transmitted at a later time after data is completely analyzed. In addition, the analysis module 220 may change a transmission period of the sensor data at a later time after the data is completely transmitted. According to one embodiment, the analysis module 220 may analyze the sensor data, and thereafter may transmit the analyzed data to another external electronic device.

According to various embodiments of the present disclosure, an electronic device may include a communication module, and a processor operatively coupled to the communication module. The processor may control to receive sensor data from at least one external electronic device through the communication module, determine an exercise type of a user who wears the at least one external electronic device based on the received sensor data, and output exercise information corresponding to the determined exercise type.

According to various embodiments of the present disclosure, the processor may control to receive the sensor data including at least one of acceleration information, angular velocity information, rotation information, geomagnetic field information, and electrocardiogram information.

According to various embodiments of the present disclosure, the processor may control to receive synchronization related information from the at least one external electronic device through the communication module.

According to various embodiments of the present disclosure, the processor may synchronize the sensor data received from the at least one external electronic device based on the synchronization related information.

According to various embodiments of the present disclosure, the processor may control to receive the synchronization related information including at least one of time stamp information, time index information, and a sampling rate of a sensor of the at least one external electronic device.

According to various embodiments of the present disclosure, the processor may classify a mobility feature of the sensor data, analyze the sensor data according to the classified mobility feature, and determine the exercise type according to a result of the analysis.

According to various embodiments of the present disclosure, the processor may determine the mobility feature of the sensor data according to a location on which the at least one external electronic device is worn, a type of the at least one external electronic device, or an input of the user.

According to various embodiments of the present disclosure, the processor may confirm a pattern of the sensor data, compare the confirmed pattern with a pre-stored pattern, and determine the exercise type according to a result of the comparison.

According to various embodiments of the present disclosure, the processor may control to output posture information, exercise count information, heart rate information, speed information, and distance information based on the exercise type.

According to various embodiments of the present disclosure, the processor may adjust at least one of a sampling rate, a transmission rate of the sensor data, and a transmission period of the sensor data according to the determined exercise type.

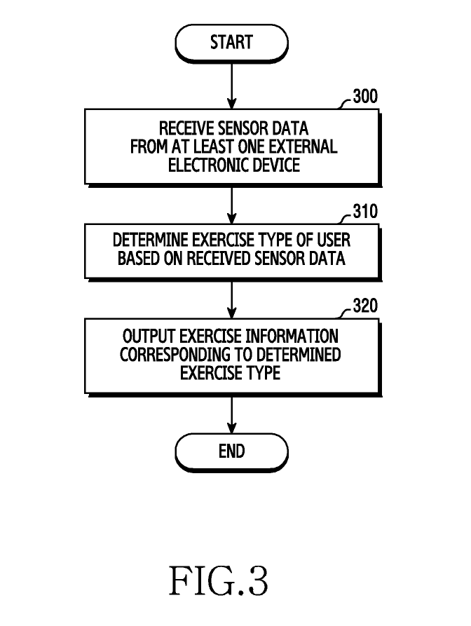

FIG. 3 is a flowchart for a method in which an electronic device outputs exercise information based on sensor data of at least one external electronic device according to an embodiment of the present disclosure.

Referring to FIG. 3, in operation 300, the electronic device (e.g., the electronic device 100) may receive sensor data from at least one external electronic device (e.g., the external electronic device 104). According to one embodiment, the at least one external electronic device may be in a state of being connected for communication with the electronic device, and may be a wearable device that can be worn on a user's body. According to one embodiment, the external electronic device may include a sensor device which is attached to the user's body to detect a user's motion. The sensor device may include an acceleration sensor, a gyro sensor, a motion sensor, a geomagnetic sensor, a rotation sensor, an ElectroCardioGram (ECG), or the like for detecting a user's posture, motion state (e.g., standstill, walking, running, stair-walking, etc.), a specific health state (e.g., a heart rate), and a specific situation (e.g., daytime, nighttime, indoor, outdoor, flooded, etc.). However, the present disclosure is not limited thereto, and thus the external electronic device may be various devices capable of detecting the user's motion. According to one embodiment, the electronic device may collect sensor data acquired by using at least one external electronic device. For example, the electronic device may collect sensor data including acceleration information, angular velocity information, rotation information, geomagnetic field information, heart rate information, or the like.

According to one embodiment, in addition to the receiving of the sensor data from the at least one external electronic device, the electronic device may receive synchronization related information from the at least one external electronic device. According to one embodiment, the synchronization related information may be time stamp information or time index information of a corresponding device. For example, the time stamp information or the time index information may include clock related information of a corresponding device or an index value or the like of a current clock. According to one embodiment, the synchronization related information may include a sampling rate, data format information, manufacturer information, or Operating System (OS) information of a corresponding sensor. For example, the synchronization related information may have various sampling rates of sensors of respective devices, and may include data format information, data unit information, or coordinate information based on X, Y, and Z-axes of each sensor.

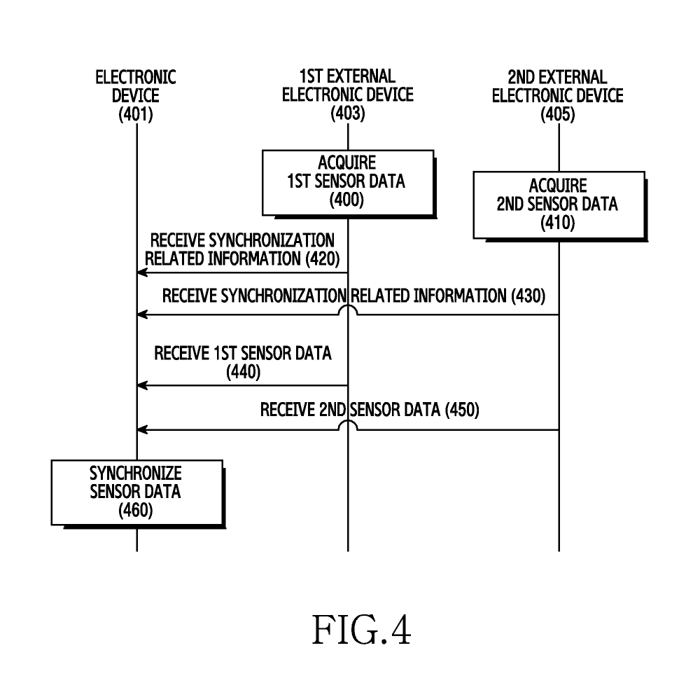

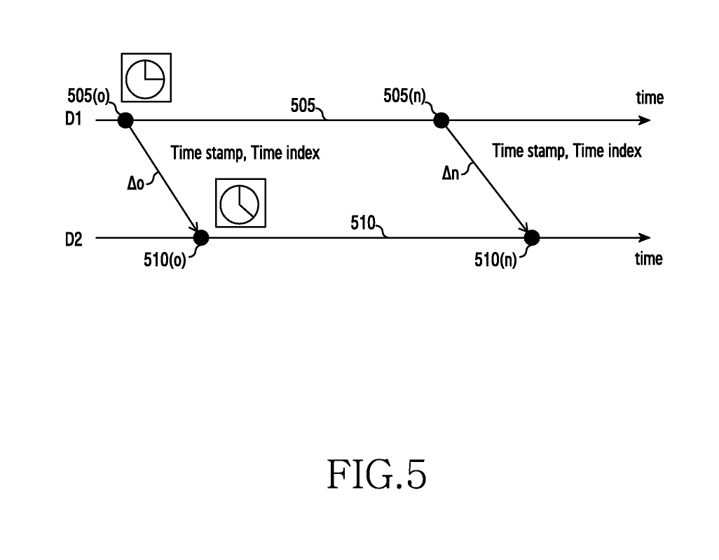

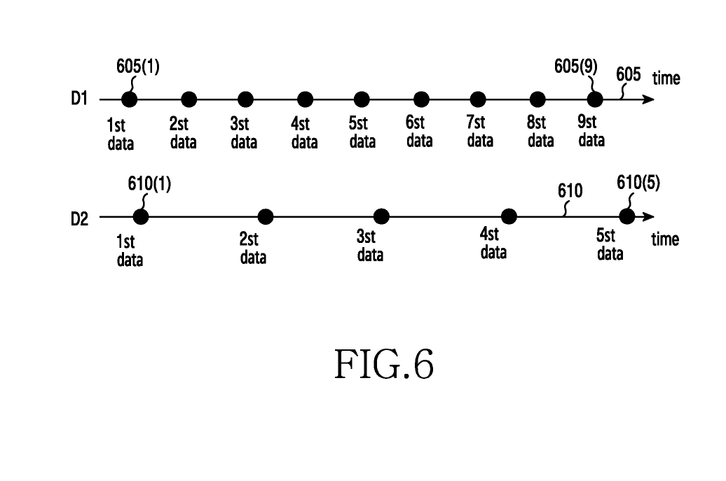

In operation 310, the electronic device may determine a user's exercise type based on received sensor data. According to one implementation, the electronic device may synchronize the sensor data based on the synchronized related information. For example, if two different external device are used, the time reference of each device may need to be synchronized so that the data can be properly correlated. According to one implementation, the electronic device may perform synchronization by utilizing a clock index value difference between respective external electronic devices. For example, the electronic device may calculate an average value by receiving a clock value several times from a first external electronic device, and thereafter may perform synchronization based on the average value. The foregoing is described in FIG. 5. According to one embodiment, as will be described in greater detail in FIG. 6, the electronic device may perform synchronization based on sampling rates on sensors of respective external electronic devices. For example, the electronic device may collect sampling rates of the first external electronic device and a second external electronic device, and may compare and analyze the collected sampling rate to perform synchronization of the sensor data. Operation 305 and synchronization will be described in FIG. 4.

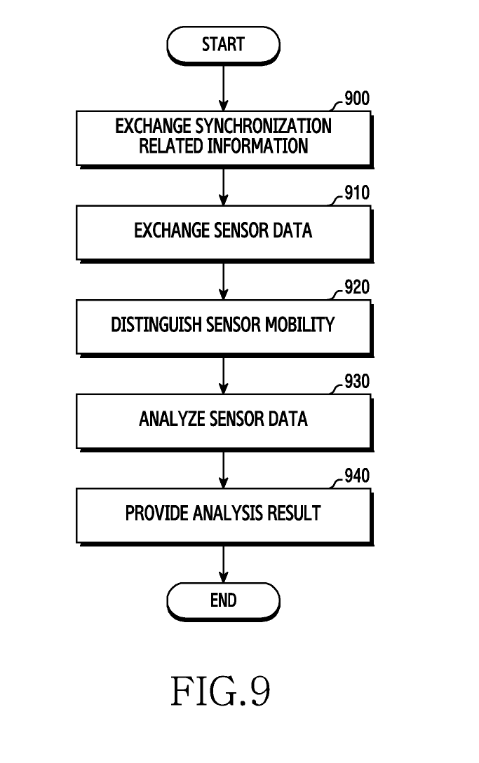

According to one embodiment, the electronic device may determine an exercise type by analyzing the synchronized sensor data. According to one embodiment, the electronic device may analyze data by utilizing the synchronized sensor data and mobility information of each sensor, and may determine the user's exercise type. According to one embodiment, the electronic device may determine the current exercise type by using a pattern of the sensor data. For example, the electronic device may have a pattern of sensor data for each exercise, and the pattern of sensor data may be classified into dynamic sensor data and static sensor data. According to one embodiment, in case of a tennis, first sensor data acquired from an external electronic device worn on a head may be classified as dynamic sensor data, and second sensor data acquired from an external electronic device worn on a wrist may be classified as static sensor data. A method of determining the exercise type by using mobility information and a sensor data pattern is described below in detail in FIG. 9.

In operation 320, the electronic device may output exercise information corresponding to the determined exercise type. According to one embodiment, the electronic device may analyze the synchronized sensor data and thereafter provide an analysis result to the user. According to one embodiment, the electronic device may recognize an exercise posture and provide exercise information (feedback) corresponding to the user's exercise type. For example, the electronic device may provide the feedback based on the exercise type on a real-time basis, and a feedback method may be determined according to the exercise type. According to one embodiment, in case of an exercise such as a sit-up during which it is difficult to see an electronic device attached to a body, the electronic device may announce a feedback based on the exercise in an auditory manner. For example, the electronic device may output the number of times of performing the sit-up as an announcement sound. According to one embodiment, in case of an exercise such as a bench press during which the electronic device attached to the body can be seen, the electronic device may announce a feedback based on the exercise in a visual manner. For example, the electronic device may output the number of times of performing the bench press as an announcement expression. According to one embodiment, in case of a golf exercise, the electronic device may provide user's gaze information and impact information as a motion related image. For example, the electronic device may output this when a specific time elapses after the user performs swing. According to one embodiment, in case of a running exercise, the electronic device may show exercise posture information, heart rate information, speed information, or the like as an image. For example, if a result of sensing the heart rate information indicates a dangerous situation, the electronic device may provide an alert through a display or may output an alert sound or may perform an emergency call. According to various embodiments, the electronic device may display exercise information corresponding to a specific exercise in various manners, and may designate a feedback scheme according to a user configuration.

Operation 300 and synchronization will now be described in more detail.

FIG. 4 is a flowchart of a method in which an electronic device synchronizes sensor data of at least one external electronic device according to an embodiment of the present disclosure.

Referring to FIG. 4, in operation 400 and operation 410, a first external electronic device 403 and a second external electronic device 405 may acquire first sensor data and second sensor data. According to one embodiment, the first external electronic device 403 or the second external electronic device 405 may be in a state of being connected for communication with an electronic device 401, and may be a wearable device that can be worn on a user's body. According to one embodiment, the first external electronic device 403 or the second external electronic device 405 may include a sensor device which is attached to the user's body to detect a user's motion. The sensor device may include an acceleration sensor, a gyro sensor, a motion sensor, a geomagnetic sensor, a rotation sensor, an ElectroCardioGram (ECG), or the like for detecting a user's posture, motion state (e.g., standstill, walking, running, stair-walking, etc.), a specific health state (e.g., a heart rate), and a specific situation (e.g., daytime, nighttime, indoor, outdoor, flooded, etc.). However, the present disclosure is not limited thereto, and thus the first external electronic device 403 and the second external electronic device 405 may be various devices capable of detecting the user's motion.

In operation 420 and operation 430, the electronic device 401 may receive synchronization related information from the first external electronic device 403 and the second external electronic device 405. According to one embodiment, the synchronization related information may be time stamp information or time index information of a corresponding device. For example, the time stamp information or the time index information may include clock related information of a corresponding device or an index value or the like of a current clock. According to one embodiment, the synchronization related information may include a sampling rate, data format information, manufacturer information, or Operating System (OS) information of a corresponding sensor. For example, the synchronization related information may have various sampling rates of sensors of respective devices, and may include data format information, data unit information, or coordinate information based on X, Y, and Z-axes of each sensor.

In operation 440 and operation 450, the electronic device 401 may receive the first sensor data and the second sensor data from the first external electronic device 403 and the second external electronic device 405. According to one embodiment, the electronic device 401 may collect sensor data acquired by using sensors of the first external electronic device 403 and the second external electronic device 405. For example, the electronic device 401 may collect sensor data including acceleration information, angular velocity information, rotation information, geomagnetic field information, heart rate information, or the like.

In operation 460, the electronic device 401 may synchronize the received sensor data. According to one embodiment, the electronic device 401 may synchronize the first sensor data and the second sensor data based on the synchronization related information received from the first external electronic device 403 and the second external electronic device 405. According to one embodiment, as shown in FIG. 5, the electronic device 401 may perform synchronization by utilizing a clock index value difference between respective devices D1 and D2. Axis 505 shows a timeline of device D1 while axis 510 shows a timeline for device D2. In one implementation, the electronic device (e.g., D2) can calculate the time difference between D1 and D2. The time difference can be calculated by, e.g., device D2 by receiving a clock value several times 505(0) . . . 505(n) from the first device D1, and comparing clock values 505(0) . . . 505(n) to corresponding times at D2, 510(0) . . . 510(n). The electronic device, e.g., D2, can calculate the time difference Δ0 . . . Δn for each corresponding pair of times. Thereafter D2 may perform synchronization based on the average value of the time differences Δ0 . . . Δn.

According to one embodiment, as shown in FIG. 6, the electronic device 401 may perform synchronization based on sampling rates on sensors of respective devices (e.g., D1 and D2). For example, the electronic device 401 may collect sampling rates of the first device D1 and the second device D2, and may compare and analyze the collected sampling rates to perform synchronization of the sensor data.

For example, axis 605 represents a timeline for electronic device D1, with sampling times 605(1) . . . 605(9). Axis 610 represents a timeline for electronic device D2, with sampling times 610(1) . . . 610(5). For instance, if the known sampling rate of electronic device D1, s, is twice the sampling rate of electronic device D2, s/2, then between each sample taken by electronic device D2, an amount of time has elapsed at electronic device D1 that is twice the sampling period.

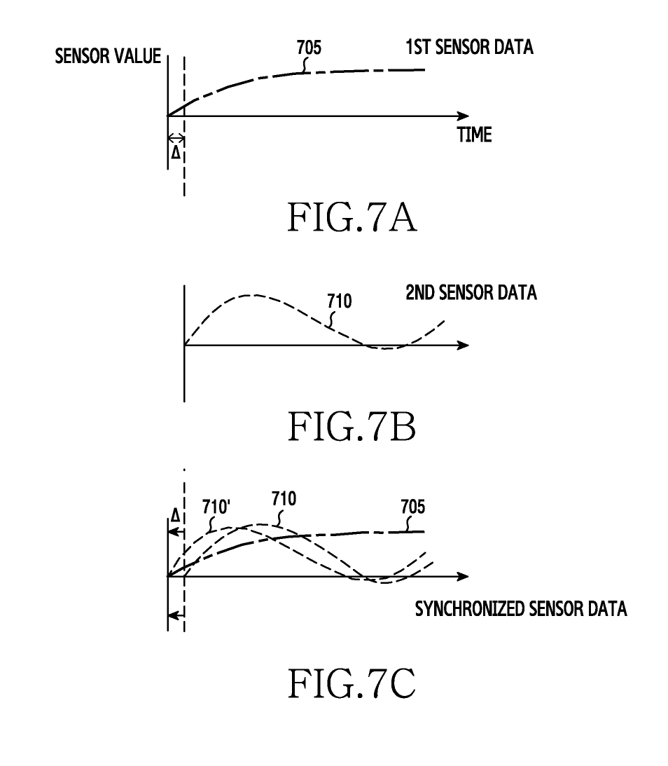

FIG. 7 illustrates synchronization of data of at least one sensor according to an embodiment of the present disclosure. According to one embodiment, synchronization (see FIG. 7C) in which first sensor data (see FIG. 7A) and second sensor data (see FIG. 7B) are synchronized in the aforementioned synchronization procedure is shown in FIG. 7. For example, an electronic device determines that second sensor data FIG. 7B, 710 is delayed 710′ with respect to first sensor data FIG. 7A, 705, by an offset Δ. Accordingly, the second sensor data 710 is shifted back by the offset Δ, resulting in synchronized data 710′.

According to various embodiments, the electronic device 401 may synchronize sensor data by using a variety of synchronization related information. Although the first external electronic device 403 and the secondexternal electronic device 405 are exclusively exemplified as at least one external electronic device, the present disclosure is not limited thereto, and thus much more external electronic devices may be included.

In certain embodiments, an external device synchronizes the sensor data and provides the synchronized data to the electronic device.

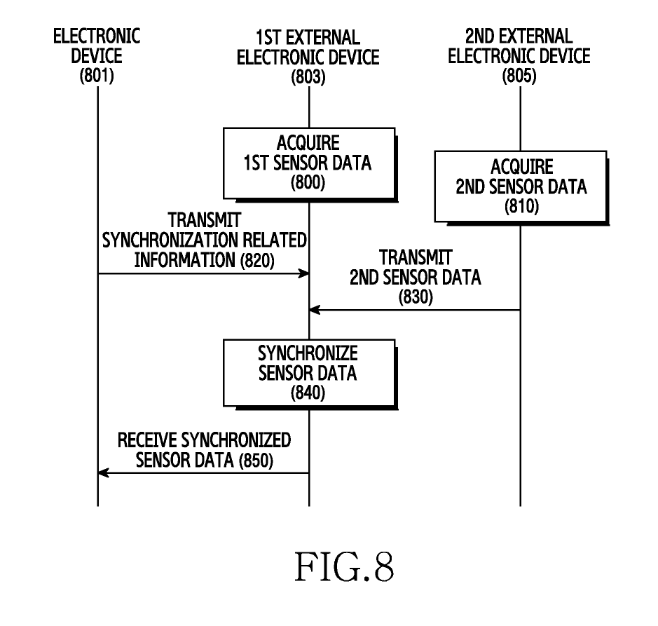

FIG. 8 is a flowchart of a method in which an electronic device receives synchronized sensor data from an external electronic device according to an embodiment of the present disclosure.

Referring to FIG. 8, in operation 800 and operation 810, a first external electronic device 803 and a second external electronic device 805 may acquire first sensor data and second sensor data. According to one embodiment, the first external electronic device 803 or the second external electronic device 805 may be in a state of being connected for communication with an electronic device 801, and may be a wearable device that can be worn on a user's body. According to one embodiment, the first external electronic device 803 or the second external electronic device 805 may include a sensor device which is attached to the user's body to detect a user's motion. The sensor device may include an acceleration sensor, a gyro sensor, a motion sensor, a geomagnetic sensor, a rotation sensor, an ElectroCardioGram (ECG), or the like for detecting a user's posture, motion state (e.g., standstill, walking, running, stair-walking), a specific health state (e.g., a heart rate), and a specific situation (e.g., daytime, nighttime, indoor, outdoor, flooded, etc.). However, the present disclosure is not limited thereto, and thus the first external electronic device 803 and the second external electronic device 805 may be various devices capable of detecting the user's motion.

In operation 820, the electronic device 801 may transmit the synchronization related information to the first external electronic device 803. The electronic device 801 may be in a state of having synchronization related information of the first external electronic device 803 and the second external electronic device 805. According to one embodiment, the synchronization related information may be time stamp information or time index information of a corresponding device. For example, the time stamp information or the time index information may include clock related information or an index value or the like of a current clock of a corresponding device. According to one embodiment, the synchronization related information may include a sampling rate, data format information, manufacturer information, or Operating System (OS) information of a corresponding sensor. For example, the synchronization related information may have various sampling rates of sensors of respective devices, and may include data format information, data unit information, or coordinate system information based on X, Y, and Z axes of each sensor.

In operation 830, the second external electronic device 805 may transmit acquired second sensor data to the first external electronic device 803. According to one embodiment, the first external electronic device 803 may collect the acquired first sensor data, and second sensor data received from the second external electronic device 805. For example, the first external electronic device 803 may collect sensor data including acceleration information, angular velocity information, rotation information, geomagnetic field information, heart rate information, or the like.

In operation 840, the first external electronic device 803 may synchronize received sensor data. According to one embodiment, the first external electronic device 803 may synchronize the first sensor data and the second sensor data based on synchronization related information received from the electronic device 801. According to one embodiment, the first external electronic device 803 may perform synchronization by utilizing a clock index value difference between respective external electronic devices. For example, the first external electronic device 803 may calculate an average value by receiving a clock value several times from the second external electronic device 805, and thereafter may perform synchronization based on the average value. According to one embodiment, the first external electronic device 803 may perform synchronization based on sampling rates on sensors of respective external electronic devices. For example, the first external electronic device 803 may collect sampling rates of the first external electronic device and the second external electronic device 805, and may compare and analyze the collected sampling rates to perform synchronization of the sensor data.

In operation 850, the electronic device 801 may receive synchronized sensor data from the first external electronic device 803.

According to various embodiments, the first external electronic device 803 may synchronize sensor data by using a variety of synchronization related information. In addition, the second external electronic device 805 other than the first external electronic device 803 may synchronize the sensor data and transmit the synchronized sensor data to the electronic device 801. According to various embodiments, although the first external electronic device 803 and the second external electronic device 805 are exclusively exemplified as at least one external electronic device, the present disclosure is not limited thereto, and thus much more external electronic devices may be included, and an external electronic device for synchronizing sensor data may be designated.

FIG. 9 is a flowchart of a method in which an electronic device analyzes sensor data of at least one external electronic device according to an embodiment of the present disclosure.

Referring to FIG. 9, in operation 900, the electronic device (e.g., the electronic device 100) may exchange synchronization related information with at least one external electronic device. According to one embodiment, the electronic device may receive the synchronization related information from the at least one external electronic device. The synchronization related information may be information required to synchronize a plurality of pieces of sensor data. According to one embodiment, the synchronization related information may be time stamp information or time index information of a corresponding device. For example, the time stamp information or the time index information may include clock related information or an index value or the like of a current clock of a corresponding device. According to one embodiment, the synchronization related information may include a sampling rate, data format information, manufacturer information, or Operating System (OS) information of a corresponding sensor. For example, the synchronization related information may have various sampling rates of sensors of respective devices, and may include data format information, data unit information, or coordinate system information based on X, Y, and Z axes of each sensor.

In operation 910, the electronic device may exchange sensor data with at least one external electronic device. According to one embodiment, the electronic device may collect sensor data acquired by using at least one external electronic device. For example, the electronic device may collect sensor data including acceleration information, angular velocity information, rotation information, geomagnetic field information, heart rate information, or the like.

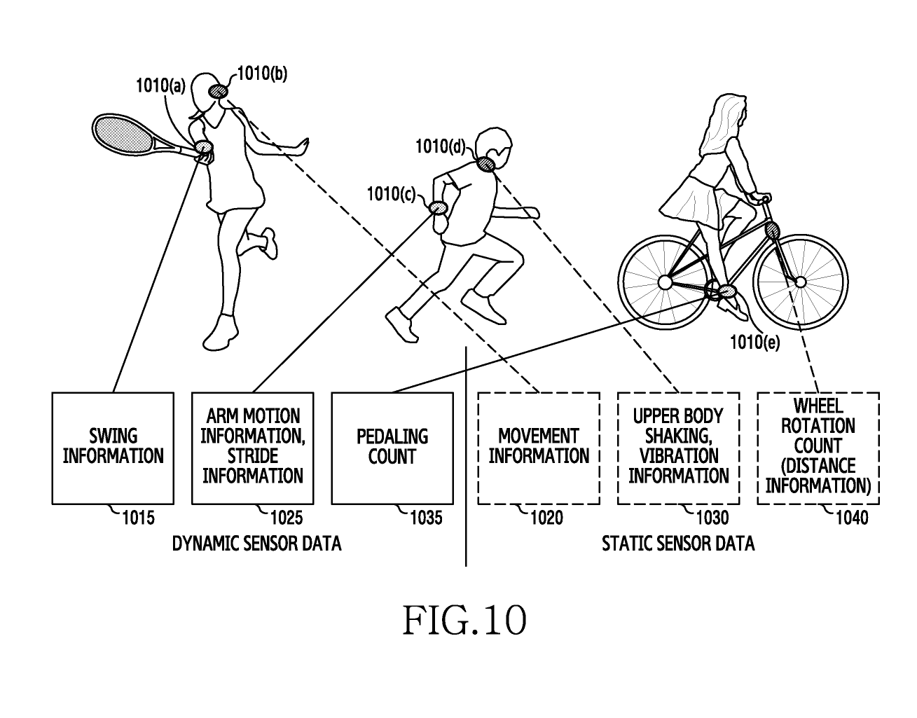

In operation 920, the electronic device may distinguish a mobility of each sensor of at least one external electronic device. According to one embodiment, each sensor may have a static mobility or dynamic mobility feature according to the mobility. For example, each sensor may have a different mobility according to a location of the sensor, and a location of the sensor may be determined according to a device type. FIG. 10 describes the mobility of wearable devices.

According to one implementation, as shown in FIG. 10, the external electronic device may include a wearable device 1010(a)-1010(e) that can be worn on various positions of a user body. The wearable device may be attached or fixed to a user's wrist 1010(a), head 1010(b), arm 1010(c), neck 1010(d), chest, belly, shoulder, leg, ankle 1010(e), or a specific position. For example, in case of a tennis, sensor data such as swing information 1015 or an impact moment acquired from an external electronic device worn on a wrist 1010(a) may be classified as dynamic sensor data, and movement information or gaze information 1020 acquired from an external electronic device worn on a head 1010(b) may be classified as static sensor data. For another example, sensor data such as arm motion information or stride information 1025 acquired from an external electronic device worn on an arm 1010(c) may be classified as dynamic sensor data, and sensor data such as upper body shaking information or vibration information 1030 acquired from an external electronic device worn on a neck 1005(d) may be classified as static sensor data. For another example, in case of a bicycle exercise, sensor data such as a pedaling count 1035 acquired in an external electronic device worn on an ankle 1005(e) may be classified as dynamic sensor data, and sensor data such as a wheel rotation count or distance information 1040 acquired from an external electronic device placed to the bicycle may be classified as static sensor data. However, the present disclosure is not limited thereto, and thus the electronic device may classify a mobility of each sensor according to various positions of the external electronic device.

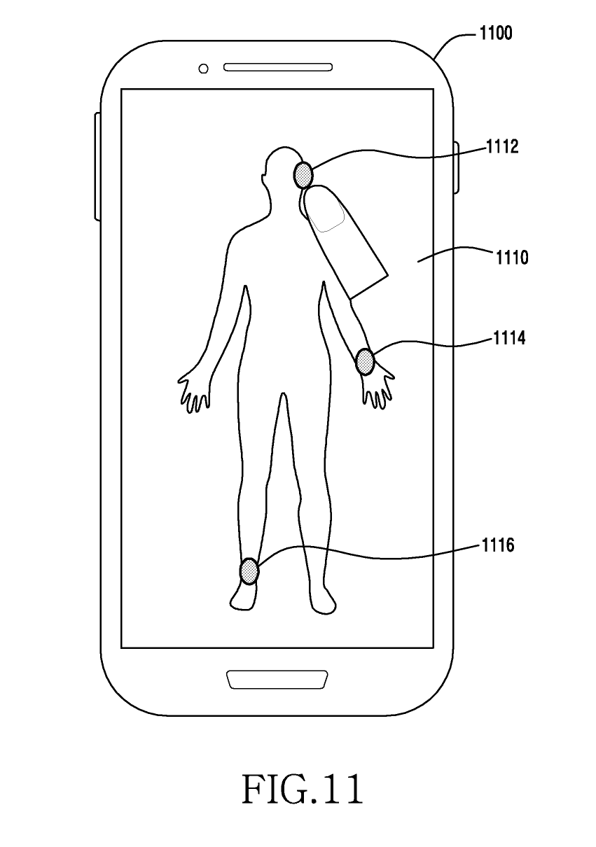

According to one embodiment, the external electronic device may be a sensor module of a pad type, not a wearable type. In this case, the electronic device may determine a sensor position according to a user input. According to one embodiment, as shown in FIG. 11, an electronic device 1100 may display a setup screen 1110 for setting a sensor position of an external electronic device when in a state of being connected with the external device or when executing a related application. For example, a user may attach a sensor to a body, and may input positions 1112, 1114, and 1116 of the attached sensor through the displayed setup screen 1110. The electronic device 1100 may change or delete the position at which the sensor is attached.

In operation 930, the electronic device may analyze the sensor data. According to one embodiment, the electronic device may synchronize the sensor data received from at least one external electronic device based on the synchronization related information. According to one embodiment, the electronic device may analyze data by utilizing the synchronized sensor data and mobility information of each sensor, and may determine the user's exercise type.

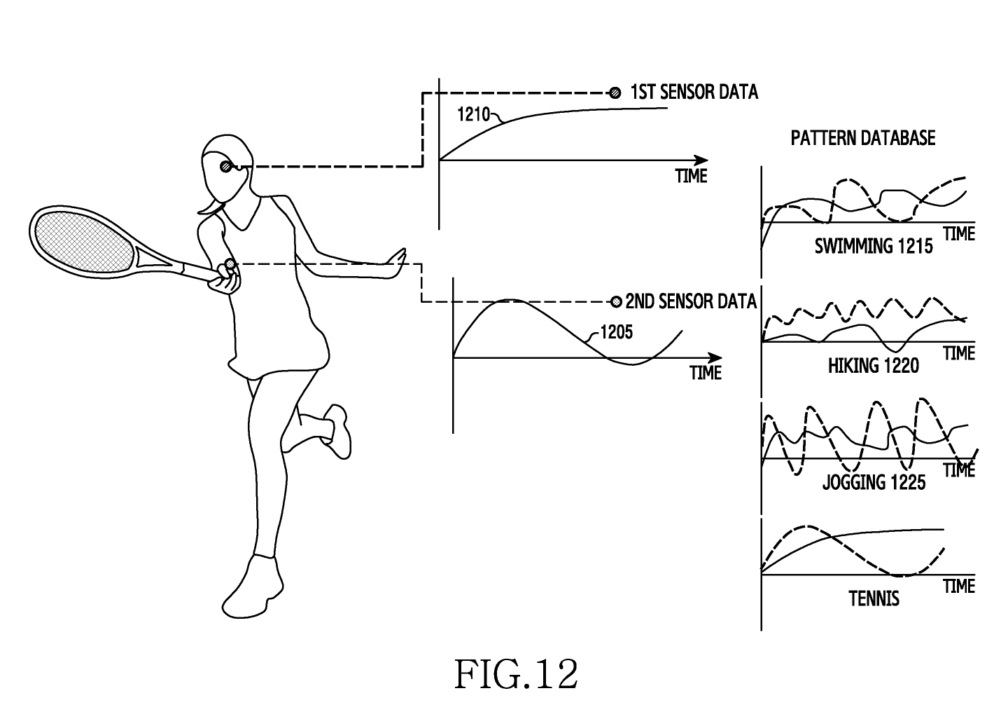

According to one embodiment, as shown in FIG. 12, an electronic device may determine a type of an exercise currently being done through a pattern of sensor data. For example, the electronic device may have a pattern of sensor data for each exercise, and the pattern of the sensor data may be classified into dynamic sensor data 1205 and static sensor data 1210. According to one embodiment, in case of a tennis, first sensor data acquired from an external electronic device worn on a head may be classified as dynamic sensor data, and second senor data acquired from an external electronic device worn on a wrist may be classified as static sensor data. For example, the first sensor data and the second sensor data may be expressed in a graph manner, and may be compared with a tennis reference graph stored in a database to determine an exercise type. In addition to the tennis, patterns of various exercise types such as swimming, hiking, jogging, or the like may be determined.

Accordingly, the electronic device 100 can store dynamic and static mobility information commonly associated with different exercises, e.g., tennis, FIG. 12, swimming 1215, hiking 1220, jogging 1225. The electronic device 100 can then compare the data received from the sensors to the dynamic and static mobility information commonly associated with each exercise, to determine the exercise that the user is engaged in.

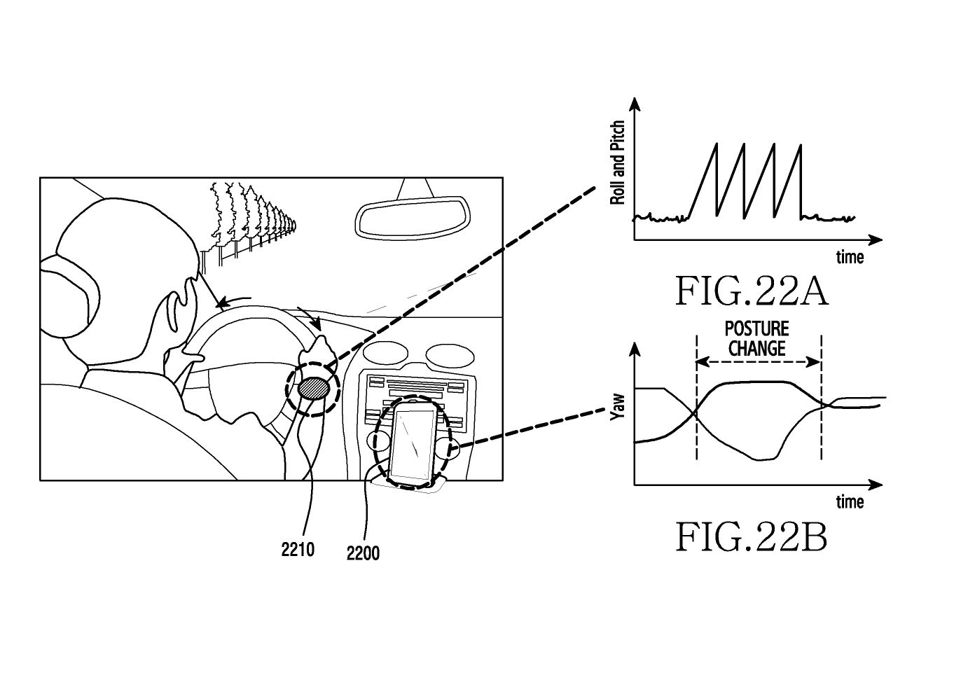

Hereinafter, a pattern of sensor data is described based on various activity types, including, for example tennis (FIG. 15), golf (FIG. 18), jump rope (FIG. 19), baseball (FIG. 20), driving (FIG. 22).

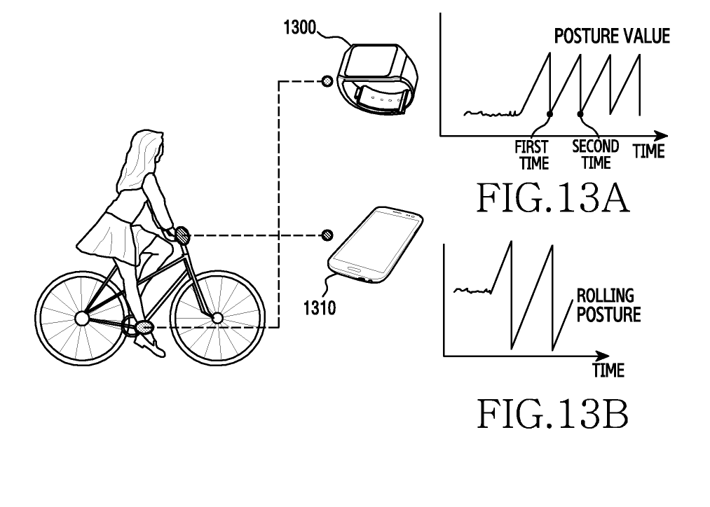

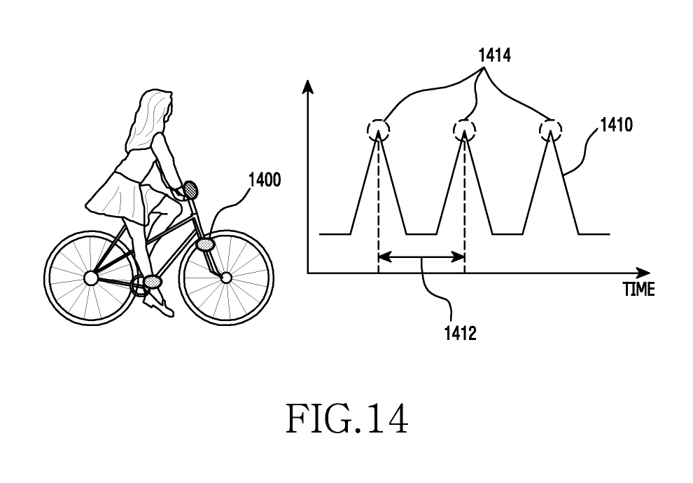

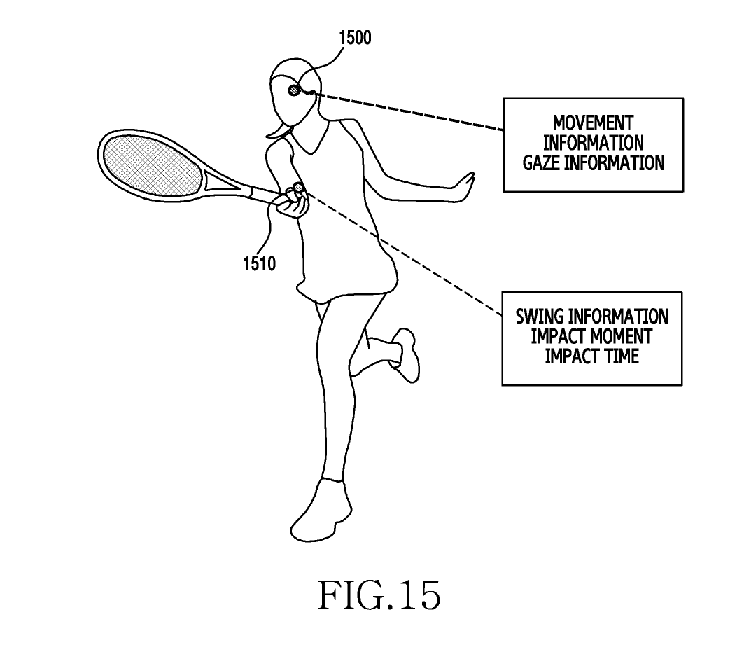

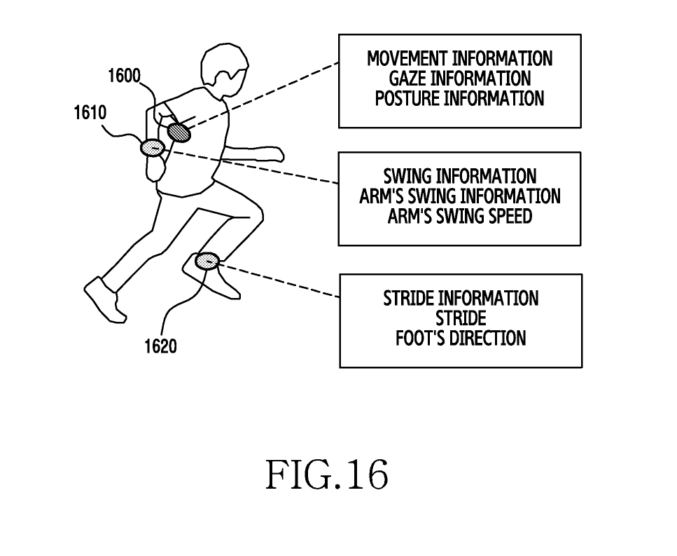

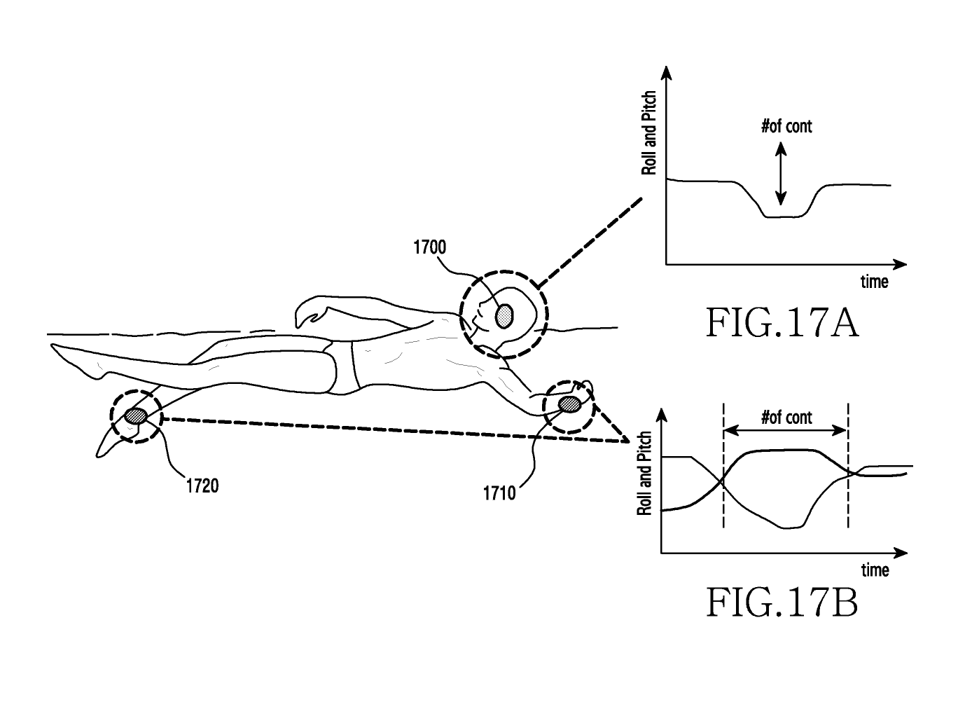

According to one embodiment, as shown in FIG. 13, an electronic device may have a pattern of detailed posture sensor data of each exercise. For example, the electronic device may recognize a posture of an exercise currently being done by a user through the pattern of the posture sensor data. According to one embodiment, the electronic device may provide a variety of exercise information by using a host terminal 1310 (i.e., a static sensor) for a bicycle exercise and a wearable device 1300 (i.e., a dynamic sensor). According to one embodiment, the electronic device may detect a pedal rotation count based on sensor data acquired through a dynamic sensor as shown in FIG. 13A, and may detect exercise motion and handling information based on sensor data acquired through a static sensor as shown in FIG. 13B. According to one embodiment, if a Global Positioning System (GPS) is lost during the bicycle exercise, the electronic device may estimate a motion more accurately by utilizing the aforementioned two pieces of sensor data, and may measure a more accurate exercise amount. According to one embodiment, as shown in FIG. 14, an electronic device may analyze a bicycle wheel rotation count and a pedaling count during the bicycle exercise. For example, a geomagnetic value based on a wheel rotation may be acquired in a state where a geomagnetic sensor 1400 is installed. The geomagnetic value may be expressed as a graph 1410, and a distorted pattern 1414 of the geomagnetic sensor may occur in every specific duration 1412.

According to one embodiment, as shown in FIG. 15, an electronic device may provide a variety of exercise information by using a first sensor 1500 (i.e., a static sensor) and a second sensor 1510 (i.e., a dynamic sensor) for a tennis exercise. According to one embodiment, the electronic device may recognize user's movement information and gaze information at a moment of hitting a ball based on sensor data acquired through the first sensor 1500, and may recognize swing information, an impact moment, an impact time, or the like based on sensor data acquired through the second sensor 1510. For example, the electronic device may analyze sensor data to distinguish a motion such as a drive posture, a cut posture, or the like during a tennis motion. In addition to the tennis, the electronic device may recognize a posture for all exercises (e.g., a table tennis, a badminton, or the like) which use a racket.

According to one embodiment, as shown in FIG. 16, an electronic device may provide a variety of exercise information by using a first sensor 1600 (i.e., a static sensor), a second sensor 1610 (i.e., a dynamic sensor), and a third sensor 1620. According to one embodiment, the electronic device may recognize user's movement information, gaze information, or posture information based on sensor data acquired through the first sensor 1600, may recognize arm's swing information, arm's swing speed, or the like, based on sensor data acquired through the second sensor 1610, and may recognize a stride, a stride direction, or the like based on sensor data acquired through the third sensor 1620.

According to one embodiment, as shown in FIG. 17, an electronic device may provide a variety of exercise information by using a first sensor 1700 (i.e., a static sensor), a second sensor 1710 (e.g., a dynamic sensor), and a third sensor 1720 (i.e., a dynamic sensor). According to one embodiment, the electronic device may detect a turn count, a head direction, or the like based on the sensor data acquired through the first sensor as shown in FIG. 17A, or may detect swimming style information such as a stroke count, a kick count, or the like based on the sensor data acquired through the second sensor 1710 and the third sensor 1720 as shown in FIG. 17B.

According to one embodiment, as shown in FIG. 18, an electronic device may provide a variety of exercise information by using a first sensor 1800 (i.e., a static sensor) and a second sensor 1810 (i.e., a dynamic sensor) for a golf exercise. According to one embodiment, the electronic device may detect gaze information based on sensor data acquired through the first sensor 1800, and may measure a swing trace, an impact moment, an impact amount, or the like based on sensor data acquired through the second sensor 1810.

According to one embodiment, as shown in FIG. 19, an electronic device may provide a variety of exercise information by using a first sensor 1900 (i.e., a static sensor) and a second sensor 1910 (i.e., a dynamic sensor) for a jumping rope exercise. According to one embodiment, the electronic device may detect jump information such as a jump count, a jump time, or the like based on sensor data acquired through the first sensor as shown in FIG. 19A, and may measure a jumping rope count or the like based on sensor data acquired through the second sensor 1910 as shown in FIG. 19B.

According to one embodiment, as shown in FIG. 20, an electronic device may provide a variety of exercise information by using a first sensor 2000 (i.e., a static sensor) and a second sensor 2010 (i.e., a dynamic sensor) for a baseball exercise. According to one embodiment, the electronic device may detect gaze information and upper body posture information based on sensor data acquired through the first sensor 2000 as shown in FIG. 20A, and may measure a swing posture, an impact moment, or the like based on sensor data acquired through the second sensor 2010 as shown in FIG. 20B.

According to various embodiments, the electronic device may determine various exercise types by using at least one dynamic sensor and at least one static sensor. In addition, the electronic device may detect a variety of posture information based on a corresponding exercise type by using the at least one dynamic sensor and at least one static sensor.

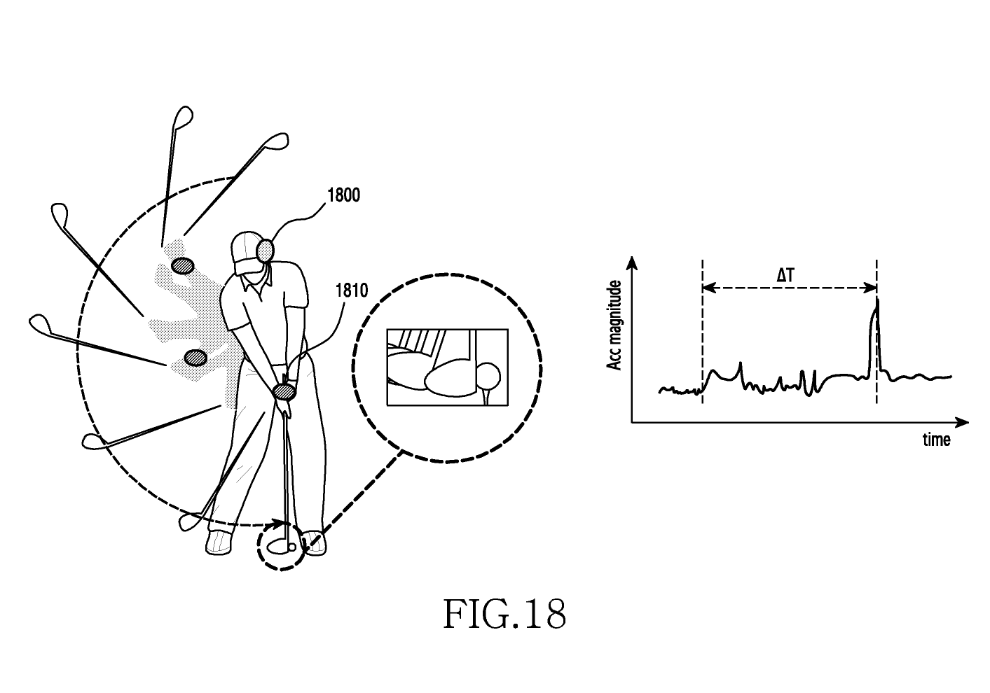

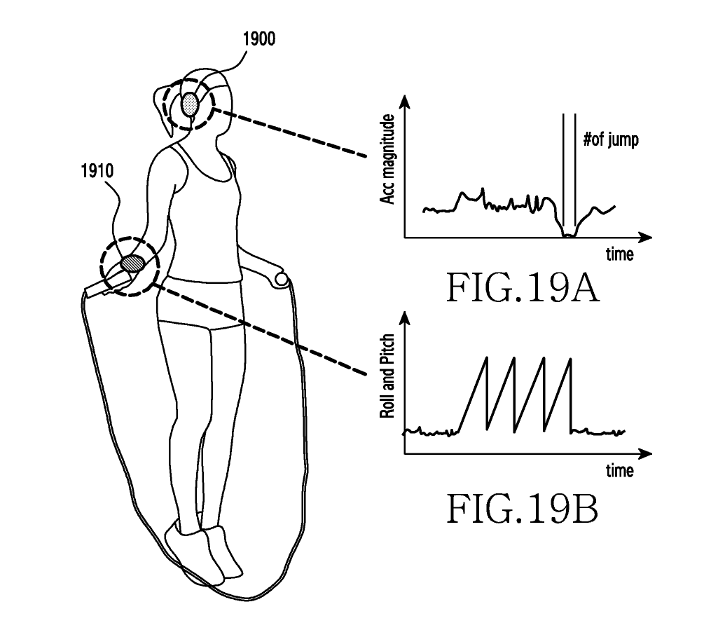

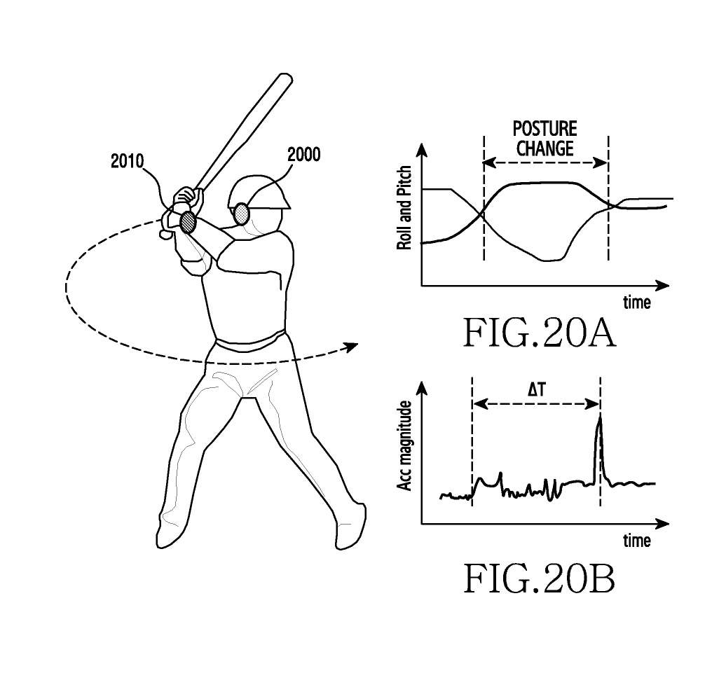

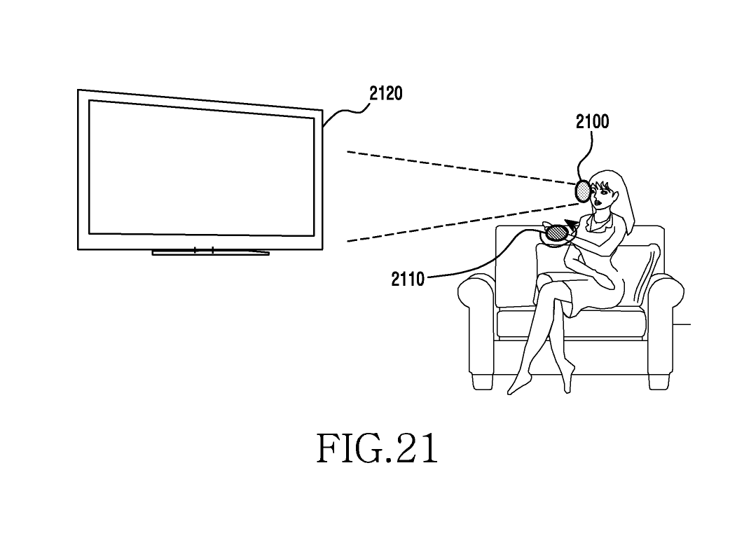

An electronic device according to various embodiments is applicable not only to a pattern of sensor data but also various fields in daily lives. For example, as shown in FIG. 21, an electronic device may control an electronic machine 2120 such as TV by using a first sensor 2100 (i.e., a static sensor) and a second sensor 2110 (i.e., a dynamic sensor). According to one embodiment, the electronic device may perform a specific command (e.g., TV ON/OFF, channel change, or the like) by using gaze information acquired through the first sensor 2100 and gesture information acquired through the second sensor 2110. For example, if a plurality of contents are present on a screen of the electronic machine 2120, the electronic device may select and control the content by using gaze information (when an area of the screen is selected) and gesture information.

For another example, as shown in FIG. 22, an electronic device may provide a variety of driving information by using a first sensor 2200 (i.e., a static sensor) and a second sensor 2210 (i.e., a dynamic sensor) for a driving motion. According to one embodiment, the electronic device may detect forward direction information based on sensor data acquired through the first sensor 2200 as shown in FIG. 22A, and may detect handling information of a vehicle based on sensor data acquired through the second sensor 2210 as shown in FIG. 22B. For example, the electronic device may use the handling information to distinguish a driver or to detect whether to perform driving.

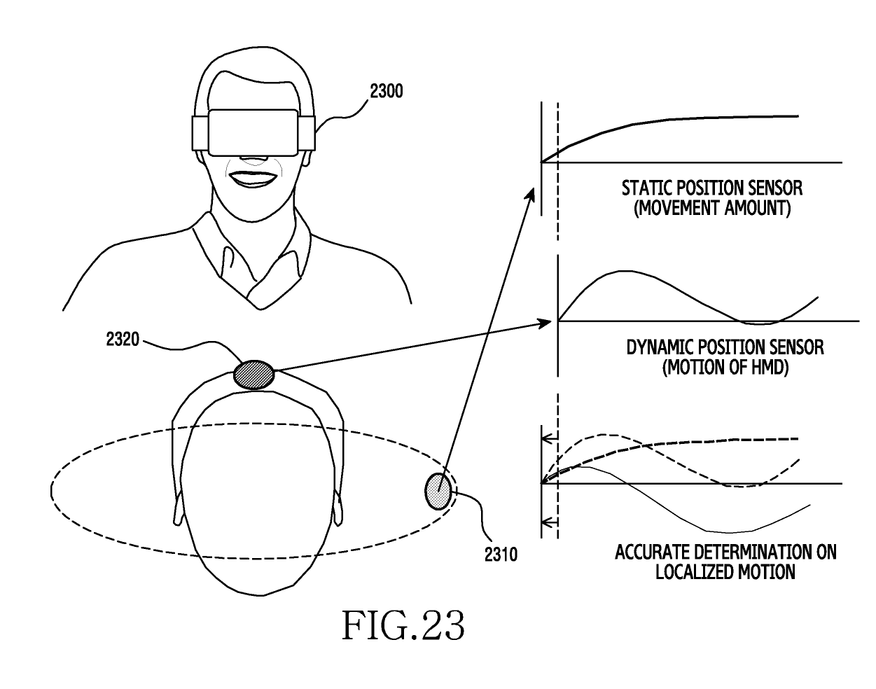

For another example, as shown in FIG. 23, in a state of wearing a Head Mount Device (HMD) 2300, an electronic device may measure a localized motion in which only a head moves whereas a body part does not move by using a first sensor 2310 (i.e., a static sensor) attached to the body and a second sensor 2320 (i.e., a dynamic sensor) attached to the HMD 2300. According to one embodiment, the electronic device may select and control a screen content of the HMD 2300 by measuring the localized motion.

In operation 940, the electronic device may analyze the synchronized sensor data and thereafter provide an analysis result to the user. According to one embodiment, the electronic device may recognize an exercise posture and provide exercise information (feedback) corresponding to the user's exercise type. For example, the electronic device may provide the feedback based on the exercise type on a real-time basis, and a feedback method may be determined according to the exercise type.

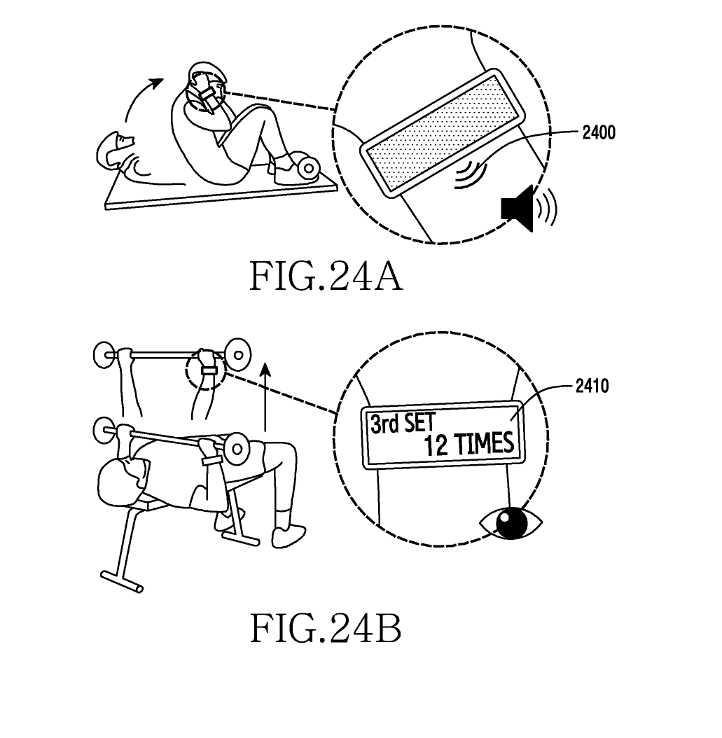

According to one embodiment, as shown in FIG. 24A, in case of an exercise such as a sit-up during which it is difficult to see an electronic device attached to a body, an electronic device may announce a feedback based on the exercise in an auditory manner. For example, the electronic device may output the number of times of performing the sit-up as an announcement sound 2400. According to one embodiment, as shown in FIG. 24B, in case of an exercise such as a bench press during which the electronic device attached to the body can be seen, an electronic device may announce a feedback based on the exercise in a visual manner. For example, the electronic device may output the number of times of performing the bench press as an announcement expression 2410.

According to one embodiment, as shown in FIG. 25, in case of a golf exercise, an electronic device may provide user's gaze information and impact information as a motion related image 2500. For example, the electronic device may output this when a specific time elapses after the user performs swing.

According to one embodiment, as shown in FIG. 26, in case of a running exercise, an electronic device may show exercise posture information, heart rate information, speed information, or the like as an image 2600. For example, if a result of sensing the heart rate information indicates a dangerous situation, the electronic device may provide an alert through a display or may output an alert sound or may perform an emergency call. According to various embodiments, the electronic device may display exercise information corresponding to a specific exercise in various manners, and may designate a feedback scheme according to a user configuration.

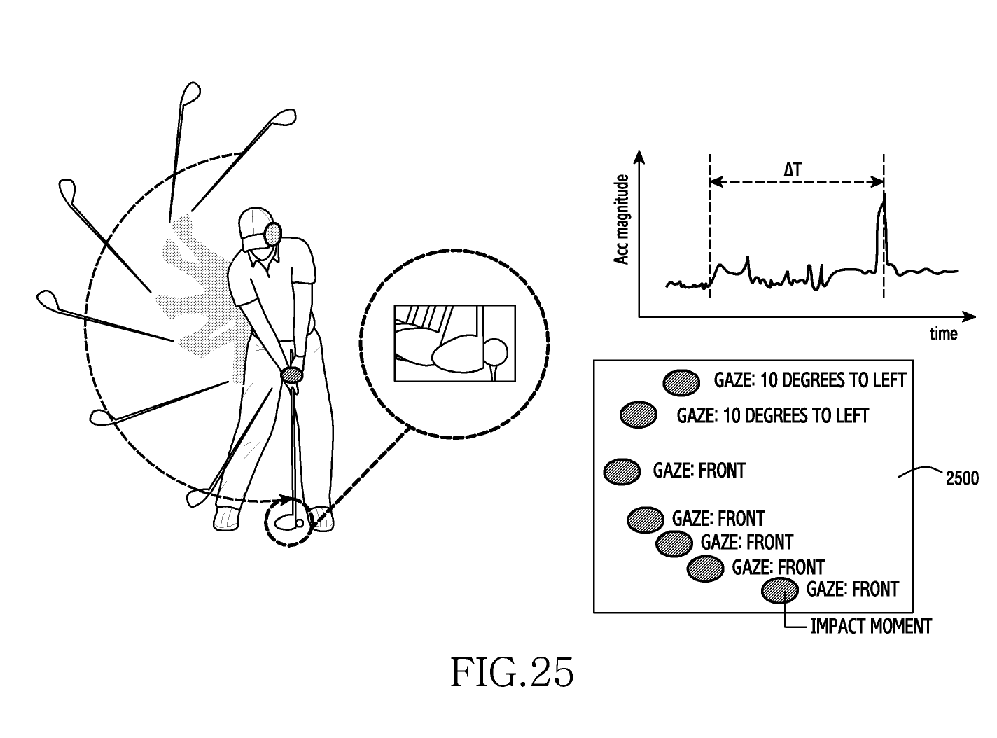

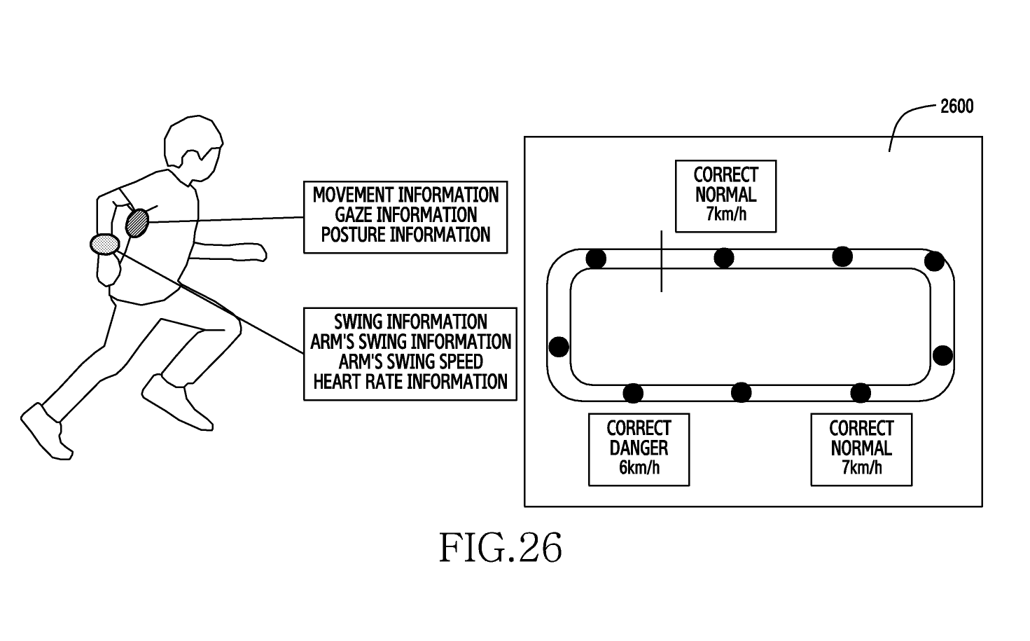

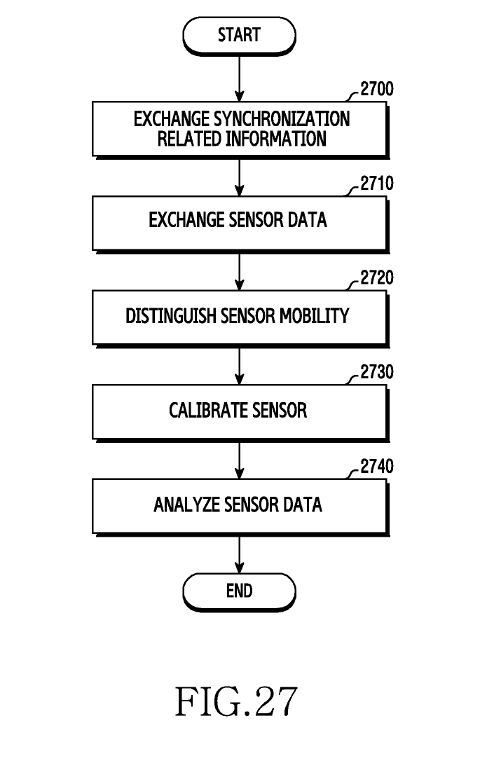

FIG. 27 is a flowchart of a method in which an electronic device synchronizes sensor data of at least one external electronic device and analyzes the synchronized sensor data according to an embodiment of the present disclosure.

Referring to FIG. 27, in operation 2700, the electronic device (e.g., the electronic device 100) may exchange synchronization related information with at least one external electronic device (e.g., the external electronic device 104). According to one embodiment, the electronic device may receive the synchronization related information from the at least one external electronic device. The synchronization related information may be information required to synchronize a plurality of pieces of sensor data. According to one embodiment, the synchronization related information may be time stamp information or time index information of a corresponding device.

In operation 2710, the electronic device may exchange sensor data with at least one external electronic device. According to one embodiment, the electronic device may collect sensor data acquired by using at least one external electronic device. For example, the electronic device may collect sensor data including acceleration information, angular velocity information, rotation information, geomagnetic field information, heart rate information, or the like.

In operation 2720, the electronic device may distinguish a mobility of each sensor of at least one external electronic device. According to one embodiment, each sensor may have a static mobility or dynamic mobility feature according to the mobility. For example, each sensor may have a different mobility according to a location of the sensor, and a location of the sensor may be determined according to a device type.

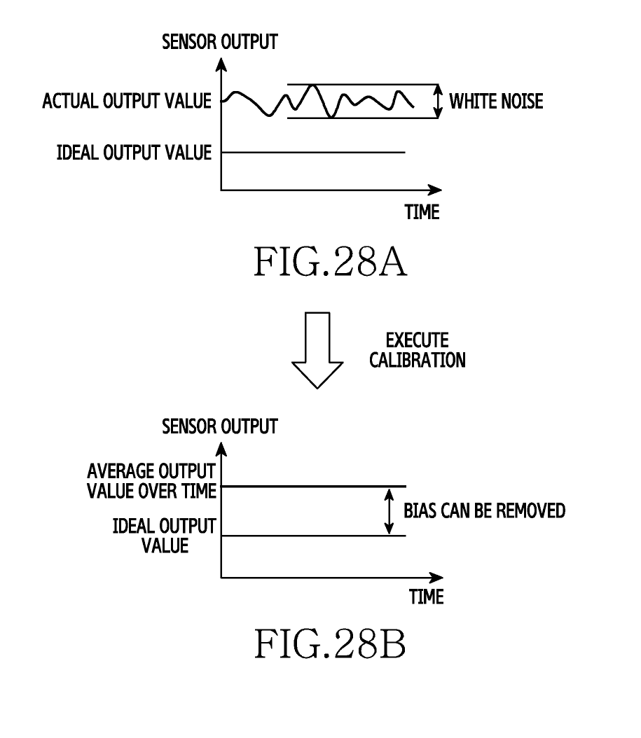

In operation 2730, the electronic device may perform calibration of sensor data. For example, the electronic device may perform the calibration operation before the sensor data is analyzed, or may perform the calibration operation when in a standstill state during daily activities. According to one embodiment, the electronic device may represent an output value of a bending sensor based on a time in a specific radius of curvature as shown in FIG. 28A. The output value has a white noise which considers an error characteristic of each sensor. According to one embodiment, as shown in FIG. 28B, the white noise may be removed by averaging an actual sensor output value including the white noise with respect to time. Such an operation may be performed to increase accuracy for analyzing synchronized exercise data to be performed next.

In operation 2740, the electronic device may analyze the sensor data. According to one embodiment, the electronic device may calibrate the sensor data received from at least one external electronic device, and thereafter may synchronize the sensor data based on the synchronization related information. According to one embodiment, the electronic device may analyze data by utilizing the synchronized sensor data and mobility information of each sensor, and may determine the user's exercise type.

In certain implementations, the electronic device can calibrate or synchronize sensors, using the determined activity type. For example, if the activity type is one involving fast motion, the electronic device can increase the sampling rate of a sensor.

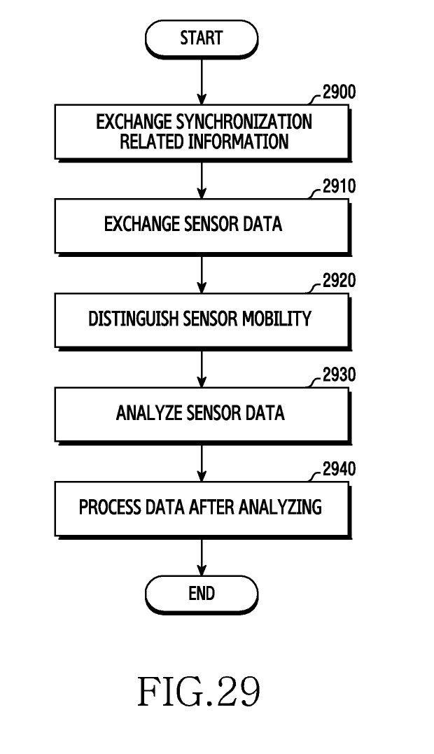

FIG. 29 is a flowchart of a method in which an electronic device synchronizes sensor data of at least one external electronic device and analyzes the synchronized sensor data according to an embodiment of the present disclosure.

Referring to FIG. 29, in operation 2900, the electronic device (e.g., the electronic device 100) may exchange synchronization related information with at least one external electronic device (e.g., the external electronic device 104). According to one embodiment, the electronic device may receive the synchronization related information from the at least one external electronic device. The synchronization related information may be information required to synchronize a plurality of pieces of sensor data. According to one embodiment, the synchronization related information may be time stamp information or time index information of a corresponding device.

In operation 2910, the electronic device may exchange sensor data with at least one external electronic device. According to one embodiment, the electronic device may collect sensor data acquired by using at least one external electronic device. For example, the electronic device may collect sensor data including acceleration information, angular velocity information, rotation information, geomagnetic field information, heart rate information, or the like.

In operation 2920, the electronic device may distinguish a mobility of each sensor of at least one external electronic device. According to one embodiment, each sensor may have a static mobility or dynamic mobility feature according to the mobility. For example, each sensor may have a different mobility according to a location of the sensor, and a location of the sensor may be determined according to a device type.

In operation 2930, the electronic device may analyze the sensor data. According to one embodiment, the electronic device may calibrate the sensor data received from at least one external electronic device, and thereafter may synchronize the sensor data based on the synchronization related information. According to one embodiment, the electronic device may analyze data by utilizing the synchronized sensor data and mobility information of each sensor, and may determine the user's exercise type.

In operation 2940, the electronic device may perform a specific function after analyzing the sensor data. According to one embodiment, the electronic device may adjust a sampling rate for the determined exercise type after analyzing the sensor data. For example, the electronic device may increase a sampling rate in a speedy exercise such as a tennis, and may decrease the sampling rate as to a relatively slow exercise such as jogging. According to one embodiment, the electronic device may adjust a data transfer rate after analyzing the sensor data. For example, the electronic device may provide control such that only a part of the sensor data is transmitted at a later time after data is completely analyzed. In addition, the electronic device may change a transmission period of the sensor data at a later time after the data is completely transmitted. According to one embodiment, the electronic device may analyze the sensor data, and thereafter may transmit the analyzed data to another external electronic device.

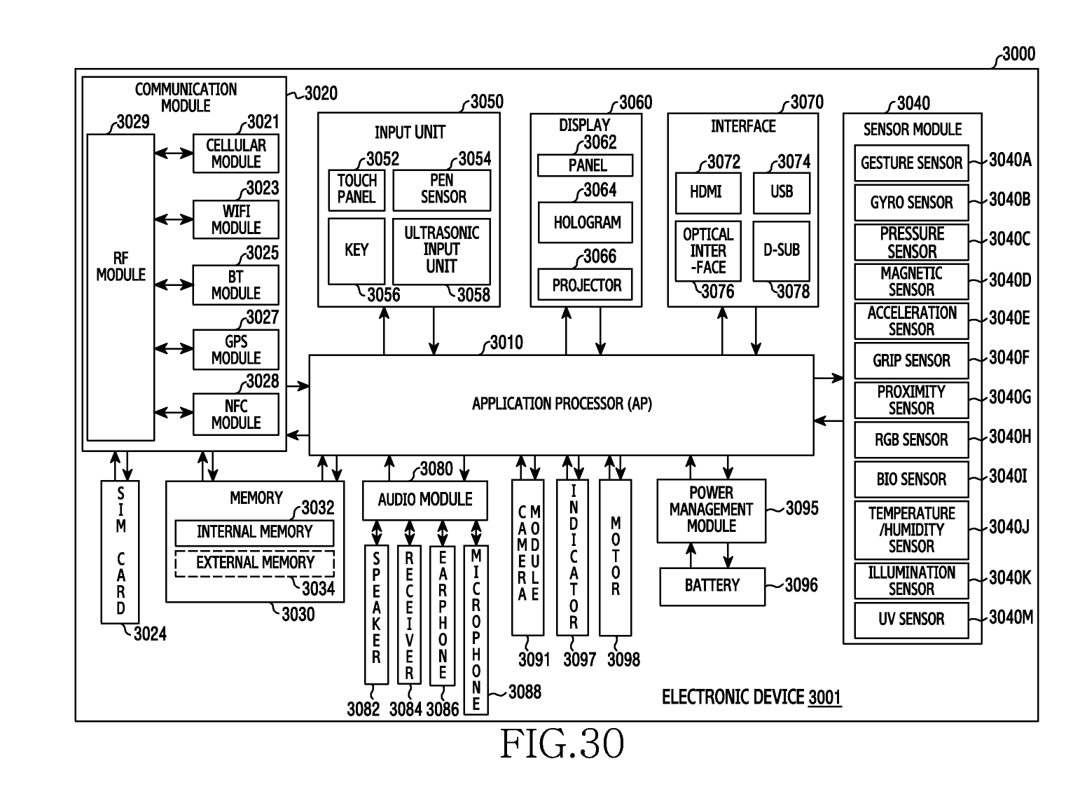

FIG. 30 illustrates a block diagram 3000 of an electronic device 3001 according to various embodiments of the present disclosure. For example, the electronic device 3001 may constitute all or some parts of the electronic device 100 of FIG. 1.

Referring to FIG. 30, the electronic device 3001 includes at least one Application Processor (AP) 3010, a communication module 3020, a Subscriber Identification Module (SIM) card 3024, a memory 3030, a sensor module 3040, an input unit 3050, a display 3060, an interface 3070, an audio module 3080, a camera module 3091, a power management module 3095, a battery 3096, an indicator 3097, and a motor 3098.

The AP 3010 may control a plurality of hardware or software constitutional elements connected to the AP 3010 by driving an operating system or an application program, and may process a variety of data including multimedia data and may perform an arithmetic operation. The AP 3010 may be implemented, for example, with a System on Chip (SoC). According to one embodiment, the AP 3010 may further include a Graphic Processing Unit (GPU, not shown).

The communication module 3020 may perform data transmission/reception in communication between other electronic devices (e.g., the electronic device 104 or the server 106) connected with the electronic device 3001 (e.g., the electronic device 100) through a network. According to one embodiment, the communication module 3020 may include a cellular module 3021, a Wi-Fi module 3023, a BlueTooth (BT) module 3025, a Global Positioning System (GPS) module 3027, a Near Field Communication (NFC) module 3028, and a Radio Frequency (RF) module 3029.

The cellular module 3021 may provide a voice call, a video call, a text service, an internet service, and the like through a communication network (e.g., LTE, LTE-A, CDMA, WCDMA, UMTS, WiBro, GSM, etc.). In addition, the cellular module 3021 may identify and authenticate the electronic device within the communication network by using a subscriber identity module (e.g., the SIM card 3024). According to one embodiment, the cellular module 3021 may perform at least some of functions that can be provided by the AP 3010. For example, the cellular module 3021 may perform at least some of multimedia control functions.

According to one embodiment, the cellular module 3021 may include a Communication Processor (CP). Further, the cellular module 3021 may be implemented, for example, with an SoC. Although constitutional elements such as the cellular module 3021 (e.g., the communication processor), the memory 3030, the power management module 3095, and the like are illustrated as separate constitutional elements with respect to the AP 3010 in FIG. 30, the AP 3010 may also be implemented such that at least one part (e.g., the cellular module 3021) of the aforementioned constitutional elements is included.

According to one embodiment, the AP 3010 or the cellular module 3021 (e.g., the communication processor) may load an instruction or data, which is received from each non-volatile memory connected thereto or at least one of different constitutional elements, to a volatile memory and may process the instruction or data. In addition, the AP 3010 or the cellular module 3021 may store data, which is received from at least one of different constitutional elements or generated by at least one of different constitutional elements, into the non-volatile memory.

Each of the WiFi module 3023, the BT module 3025, the GPS module 3027, and the NFC module 3028 may include, for example, a processor for processing data transmitted/received through a corresponding module. Although the cellular module 3021, the WiFi module 3023, the BT module 3025, the GPS module 3027, and the NFC module 3028 are illustrated in FIG. 30 as separate blocks, according to one embodiment, at least some (e.g., two or more) of the cellular module 3021, the WiFi module 3023, the BT module 3025, the GPS module 3027, and the NFC module 3028 may be included in one Integrated Chip (IC) or IC package. For example, at least some of processors corresponding to the cellular module 3021, the WiFi module 3023, the BT module 3025, the GPS module 3027, and the NFC module 3028 (e.g., a communication processor corresponding to the cellular module 3021 and a WiFi processor corresponding to the WiFi module 3023) may be implemented with an SoC.

The RF module 3029 may serve to transmit/receive data, for example, to transmit/receive an RF signal. Although not shown, the RF module 3029 may include, for example, a transceiver, a Power Amp Module (PAM), a frequency filter, a Low Noise Amplifier (LNA), and the like. In addition, the RF module 3029 may further include a component for transmitting/receiving a radio wave on a free space in wireless communication, for example, a conductor, a conducting wire, and the like. Although it is illustrated in FIG. 30 that the cellular module 3021, the WiFi module 3023, the BT module 3025, the GPS module 3027, and the NFC module 3028 share one RF module 3029, according to one embodiment, at least one of the cellular module 3021, the WiFi module 3023, the BT module 3025, the GPS module 3027, the NFC module 3028 may transmit/receive an RF signal via a separate RF module.

The SIM card 3024 may be a card in which a SIM is implemented, and may be inserted to a slot formed at a specific location of the electronic device. The SIM card 3024 may include unique identification information (e.g., an Integrated Circuit Card IDentifier (ICCID)) or subscriber information (e.g., an International Mobile Subscriber Identity (IMSI)).

The memory 3030 (e.g., the memory 130) may include an internal memory 3032 or an external memory 3034. The internal memory 3032 may include, for example, at least one of a volatile memory (e.g., a Dynamic RAM (DRAM), a Static RAM (SRAM), a Synchronous Dynamic RAM (SDRAM), etc.) or a non-volatile memory (e.g., a One Time Programmable ROM (OTPROM), a Programmable ROM (PROM), an Erasable and Programmable ROM (EPROM), an Electrically Erasable and Programmable ROM (EEPROM), a Mask ROM, a Flash ROM, a NAND flash memory, a NOR flash memory, etc.).

According to one embodiment, the internal memory 3032 may be a Solid State Drive (SSD). The external memory 3034 may further include a flash drive, and may further include, for example, Compact Flash (CF), Secure Digital (SD), Micro Secure Digital (Micro-SD), Mini Secure digital (Mini-SD), extreme Digital (xD), memory stick, and the like. The external memory 3034 may be operatively coupled to the electronic device 3001 via various interfaces. According to one embodiment, the electronic device 3001 may further include a storage unit (or a storage medium) such as a hard drive.

The sensor module 3040 may measure a physical quantity or detect an operation state of the electronic device 3001, and thus may convert the measured or detected information into an electric signal. The sensor module 3040 may include, for example, at least one of a gesture sensor 3040A, a gyro sensor 3040B, a pressure sensor 3040C, a magnetic sensor 3040D, an acceleration sensor 3040E, a grip sensor 3040F, a proximity sensor 3040G, a color sensor 3040H (e.g., a Red, Green, Blue (RGB) sensor), a bio sensor 3040I, a temperature/humidity sensor 3040J, an illumination sensor 3040K, and an Ultra Violet (UV) sensor 3040M. Additionally or alternatively, the sensor module 3040 may include, for example, an E-nose sensor (not shown), an ElectroMyoGraphy (EMG) sensor (not shown), an ElectroEncephaloGram (EEG) sensor (not shown), an ElectroCardioGram (ECG) sensor (not shown), an Infra Red (IR) sensor (not shown), an iris sensor (not shown), a fingerprint sensor (not shown), and the like. The sensor module 3040 may further include a control circuit for controlling at least one or more sensors included therein.

The input device 3050 may include a touch panel 3052, a (digital) pen sensor 3054, a key 3056, or an ultrasonic input unit 3058. The touch panel 3052 may recognize a touch input, for example, by using at least one of an electrostatic type, a pressure-sensitive type, and an ultrasonic type. The touch panel 3052 may further include a control circuit. In case of the electrostatic type, not only a physical contact but also a proximity recognition is possible. The touch penal 3052 may further include a tactile layer. In this case, the touch panel 3052 may provide the user with a tactile reaction.

The (digital) pen sensor 3054 may be implemented, for example, by using the same or similar method of receiving a touch input of the user or by using an additional sheet for recognition. The key 3056 may be, for example, a physical button, an optical key, a keypad, or a touch key. The ultrasonic input unit 3058 is a device by which the electronic device 3001 detects a sound wave through a microphone 3088 by using a pen which generates an ultrasonic signal, and is a device capable of radio recognition. According to one embodiment, the electronic device 3001 may use the communication module 3020 to receive a user input from an external device (e.g., a computer or a server) connected thereto.

The display 3060 may include a panel 3062, a hologram 3064, or a projector 3066. The panel 3062 may be, for example, a Liquid-Crystal Display (LCD), an Active-Matrix Organic Light-Emitting Diode (AM-OLED), and the like. The panel 3062 may be implemented, for example, in a flexible, transparent, or wearable manner. The panel 3062 may be constructed as one module with the touch panel 3052. The hologram 3064 may use an interference of light and show a stereoscopic image in the air. The projector 3066 may display an image by projecting a light beam onto a screen. The screen may be located, for example, inside or outside the electronic device 3001. According to one embodiment, the display 3060 may further include a control circuit for controlling the panel 3062, the hologram 3064, or the projector 3066.

The interface 3070 may include, for example, a High-Definition Multimedia Interface (HDMI) 3072, a Universal Serial Bus (USB) 3074, an optical communication interface 3076, or a D-subminiature (D-sub) 3078. The interface 3070 may be included, for example, in the communication interface 160 of FIG. 1. Additionally or alternatively, the interface 3070 may include, for example, a Mobile High-definition Link (MHL) interface, a Secure Digital (SD)/Multi-Media Card (MMC) interface, or an Infrared Data Association (IrDA) standard interface.

The audio module 3080 may bilaterally convert a sound and electronic signal. The audio module 3080 may convert sound information which is input or output, for example, through a speaker 3082, a receiver 3084, an earphone 3086, the microphone 3088, and the like.

The camera module 3091 is a device for image and video capturing, and according to one embodiment, may include one or more image sensors (e.g., a front sensor or a rear sensor), a lens (not shown), an Image Signal Processor (ISP) (not shown), or a flash (not shown, e.g., LED or xenon lamp).

The power management module 3095 may manage a power of the electronic device 3001. Although not shown, the power management module 3095 may include, for example, a Power Management Integrated Circuit (PMIC), a charger Integrated Circuit (IC), or a battery fuel gauge. The PMIC may be placed, for example, inside an IC or SoC semiconductor.

Charging may be classified into wired charging and wireless charging. The charger IC may charge a battery, and may avoid an over-voltage or over-current flow from a charger. According to one embodiment, the charger IC may further include a charger IC for at least one of the wired charging and the wireless charging. The wireless charging may be classified into, for example, a magnetic resonance type, a magnetic induction type, and an electromagnetic type. An additional circuit for the wireless charging, for example, a coil loop, a resonant circuit, a rectifier, and the like, may be added.

The battery gauge may measure, for example, a residual quantity of the battery 3096 and a voltage, current, and temperature during charging. The battery 3096 may store or generate an electricity, and may supply a power to the electronic device 3001 by using the stored or generated electricity. For example, the battery 3096 may include a rechargeable battery or a solar battery.

The indicator 3097 may indicate a specific state, for example, a booting state, a message state, a charging state, and the like, of the electronic device 3001 or a part thereof (e.g., the AP 3010). The motor 3098 may convert an electric signal into a mechanical vibration. Although not shown, the electronic device 3001 may include a processing unit (e.g., a GPU) for supporting a mobile TV. The processing unit for supporting the mobile TV may process media data according to a protocol of, for example, Digital Multimedia Broadcasting (DMB), Digital Video Broadcasting (DVB), media flow, and the like.

Each of the aforementioned constitutional elements of the electronic device according to various embodiments of the present disclosure may consist of one or more components, and names thereof may vary depending on a type of electronic device. The electronic device according to various embodiments of the present disclosure may include at least one of the aforementioned constitutional elements. Some of the constitutional elements may be omitted, or additional other constitutional elements may be further included. In addition, some of the constitutional elements of the electronic device according to various embodiments of the present disclosure may be combined and constructed as one entity, so as to equally perform functions of corresponding constitutional elements before combination

According to various embodiments, a sensor data processing method and an electronic device thereof analyze exercise information based on an attribute of sensor data, and provide exercise information corresponding to an exercise type, thereby improving user's health by guiding a correct posture.

A term “module” used in various embodiments of the present disclosure may imply a unit including, for example, one of hardware, software, and firmware or a combination of two or more of them. The “module” may be interchangeably used with a term such as a unit, a logic, a logical block, a component, a circuit, and the like. The “module” may be a minimum unit of an integrally constituted component or may be a part thereof. The “module” may be a minimum unit for performing one or more functions or may be a part thereof. The “module” may be mechanically or electrically implemented. For example, the “module” according to various embodiments of the present disclosure may include at least one of an Application-Specific Integrated Circuit (ASIC) chip, a Field-Programmable Gate Arrays (FPGAs), and a programmable-logic device, which are known or will be developed and which perform certain operations.

According to various embodiments, at least some parts of a device (e.g., modules or functions thereof) or method (e.g., operations) may be implemented with an instruction stored in a computer-readable storage media for example. If the instruction is executed by one or more processors (e.g., the processor 120), the one or more processors may perform a function corresponding to the instruction. The computer-readable storage media may be, for example, the memory 130. At least some parts of the programming module may be implemented (e.g., executed), for example, by the processor 120. At least some parts of the programming module may include modules, programs, routines, sets of instructions, processes, and the like, for performing one or more functions.

The computer readable recording medium may be a hardware device configured particularly to store and perform a program instruction (e.g., program module), for example, a hard disk, a magnetic medium such as a floppy disc and a magnetic tape, an optical storage medium such as a Compact Disc-ROM (CD-ROM) or a Digital Versatile Disc (DVD), a magnetic-optic medium such as a floptical disc, a Read Only Memory (ROM), a Random Access Memory (RAM), a flash memory, and the like. An example of the program instruction includes not only a machine language created by a compiler but also a high-level language executable by a computer by using an interpreter or the like. The aforementioned hardware device may be configured to operate as one or more software modules to perform the operation of various embodiments of the present disclosure, and the other way around is also possible.

The module or programming module according to various embodiments of the present disclosure may further include at least one or more constitutional elements among the aforementioned constitutional elements, or may omit some of them, or may further include additional other constitutional elements. Operations performed by a module, programming module, or other constitutional elements of the present disclosure may be executed in a sequential, parallel, repetitive, or heuristic manner. In addition, some of the operations may be executed in a different order or may be omitted, or other operations may be added.

According to various embodiments, in a storage medium for storing instructions, when the instructions are executed by at least one processor, the at least one processor may be allowed to perform at least one operation including receiving sensor data from at least one external electronic device, determining an exercise type of a user who wears the at least one external electronic device based on the received sensor data, and outputting exercise information corresponding to the determined exercise type.

While various embodiments of the present disclosure have been shown and described with reference to certain preferred embodiments thereof, it will be understood by those skilled in the art that various changes in form and details may be made therein without departing from the spirit and scope of the embodiments of the present disclosure as defined by the appended claims. Therefore, the scope of the various embodiments of the present disclosure is defined not by the detailed description of the various embodiments of the present disclosure but by the appended claims, and all differences within the scope will be construed as being included in the various embodiments of the present disclosure.

What is claimed is:

1. A method in an electronic device, the method comprising:
receiving sensor data from at least one external electronic device communicating with the electronic device;
determining an exercise type of a user who wears the at least one external electronic device based on the received sensor data; and
outputting exercise information corresponding to the determined exercise type.

2. The method of claim 1, wherein the sensor data comprises at least one of acceleration information, angular velocity information, rotation information, geomagnetic field information, and electrocardiogram information.

3. The method of claim 1, further comprising receiving synchronization related information from the at least one external electronic device.

4. The method of claim 3, further comprising synchronizing the sensor data received from the at least one external electronic device based on the synchronization related information.

5. The method of claim 3, wherein the synchronization related information comprises at least one of time stamp information, time index information, and a sampling rate of a sensor of the at least one external electronic device.

6. The method of claim 1, wherein the determining of the exercise type comprises:
classifying a mobility feature of the sensor data;
analyzing the sensor data according to the classified mobility feature; and
determining the exercise type according to a result of analyzing the sensor data according to the classified mobility feature.

7. The method of claim 6, wherein the mobility feature of the sensor data is determined according to a location on which the at least one external electronic device is worn, a type of the at least one external electronic device, or an input of the user.

8. The method of claim 1, wherein the determining of the exercise type comprises:
confirming a pattern of the sensor data;
comparing the confirmed pattern with a pre-stored pattern; and
determining the exercise type according to a result of comparing the confirmed patter with a pre-stored pattern.

9. The method of claim 1, wherein the outputting of the exercise information comprises outputting at least one of posture information, exercise count information, heart rate information, speed information, and distance information based on the exercise type.

10. The method of claim 1, further comprising, after the determining of the exercise type, adjusting at least one of a sampling rate, a transmission rate of the sensor data, and a transmission period of the sensor data according to the determined exercise type.

11. An electronic device comprising:
a communication module; and
at least one processor configured to control to:
receive sensor data from at least one external electronic device communicating with the electronic device through the communication module;
determine an exercise type of a user who wears the at least one external electronic device based on the received sensor data; and
output exercise information corresponding to the determined exercise type.

12. The electronic device of claim 11, wherein the at least one processor is further configured to control to receive the sensor data comprising at least one of acceleration information, angular velocity information, rotation information, geomagnetic field information, and electrocardiogram information.

13. The electronic device of claim 11, wherein the at least one processor is further configured to control to receive synchronization related information from the at least one external electronic device through the communication module.

14. The electronic device of claim 13, wherein the at least one processor is further configured to control to synchronize the sensor data received from the at least one external electronic device based on the synchronization related information.

15. The electronic device of claim 13, wherein the at least one processor is further configured to control to receive the synchronization related information comprising at least one of time stamp information, time index information, and a sampling rate of a sensor of the at least one external electronic device.

16. The electronic device of claim 11, wherein the at least one processor is further configured to control to classify a mobility feature of the sensor data, analyze the sensor data according to the classified mobility feature, and determine the exercise type according to a result of analyzing the sensor data according to the classified mobility feature.

17. The electronic device of claim 16, wherein the at least one processor is further configured to control to determine the mobility feature of the sensor data according to a location on which the at least one external electronic device is worn, a type of the at least one external electronic device, or an input of the user.

18. The electronic device of claim 11, wherein the at least one processor is further configured to control to confirm a pattern of the sensor data, compares the confirmed pattern with a pre-stored pattern, and determine the exercise type according to a result of the comparison of the confirmed pattern with the pre-stored pattern.

19. The electronic device of claim 11, wherein the at least one processor is further configured to output at least one of posture information, exercise count information, heart rate information, speed information, and distance information based on the exercise type.

20. The electronic device of claim 11, wherein the at least one processor is further configured to control to adjust at least one of a sampling rate, a transmission rate of the sensor data, and a transmission period of the sensor data according to the determined exercise type.

21. The electronic device of claim 11, wherein the at least one processor comprises a sensor data processing module.

22. An electronic device comprising:
a memory storing mobility data for a plurality of activity types;
a communication interface configured to receive sensor data from at least one external electronic device; and
at least one processor configured to determine a particular one of the plurality of activity types by comparing the sensor data from the at least one external device or processed sensor data from the at least one external device to the mobility data for the plurality of activity types.

23. The electronic device of claim 22, wherein the at least one processor performs at least one of:
synchronizing the sensor data from the at least one external electronic device; and
classifying a mobility feature of the sensor data, thereby resulting in the processed sensor data.

24. The electronic device of claim 23, wherein:
the at least one external electronic device comprises a first external electronic device and a second external electronic device;
the sensor data comprises sensor data from the first external electronic device and sensor data from the second electronic device; and
synchronizing the sensor data comprises synchronizing the sensor data from the first external electronic device and the sensor data from the second external electronic device.
